(19) United States
(12) Patent Application Publication
(10) Pub. No.: US 2017/0347771 A1
(43) Pub. Date: Dec. 7, 2017

(54) MULTI-COMPONENT PACKAGING UNIT AND METHOD FOR REDUCTIVELY DECOLORING DYED KERATIN FIBERS

(71) Applicant: Henkel AG & Co. KGaA, Duesseldorf (DE)

(72) Inventors: Juergen Schoepgens, Schwalmtal (DE); Burkhard Mueller, Duesseldorf (DE); Constanze Neuba, Grevenbroich (DE)

(73) Assignee: Henkel AG & Co. KGaA, Duesseldorf (DE)

(21) Appl. No.: 15/609,024

(22) Filed: May 31, 2017

(30) Foreign Application Priority Data
Jun. 7, 2016 (DE) 102016209981.7

(51) Int. Cl. A45D 40/00; A45D 34/00; A61K 8/20; A61K 8/46; A61Q 5/08; A61K 8/34

(52) U.S. Cl. CPC A45D 40/00; A61Q 5/08; A61K 8/342; A61K 8/20; A61K 8/46; A45D 34/00; A45D 2040/0093; A61K 2800/87

(57) ABSTRACT

Multi-component packaging units for reductively decoloring dyed keratin fibers and methods for reductively decoloring dyed keratin fibers are provided herein. In an embodiment, a multi-component packaging unit for reductively decoloring dyed keratin fibers includes a container (A) that includes a cosmetic agent (a) and a container (B) that includes a cosmetic agent (b), which are produced separately from each other. Agent (a) in container (A) includes one or more sulfur-containing reductants. Agent (b) in container (B) includes one or more metal salts chosen from the group of tin(II) salts, iron(II) salts, manganese(II) salts, titanium(II) salts, or titanium(III) salts.

### CROSS-REFERENCE TO RELATED APPLICATION

This application claims priority to German Patent Application No. 10 2016 209 981.7, filed Jun. 7, 2016, which is incorporated herein by reference in its entirety.

### TECHNICAL FIELD

The present disclosure lies in the field of cosmetics and relates to a multi-component packaging unit (kits of parts) for reductively decoloring dyed keratin fibers, which comprises the containers (A) and (B), which are produced separately from each other. Container (A) contains a cosmetic agent (a), which contains at least one sulfur-containing reductant. Container (B) contains a cosmetic agent (b), which contains one or more metal salts from the group of the tin(II) salts, the iron(II) salts, the manganese(II) salts, the titanium(II) salts, and the titanium(III) salts.

### BACKGROUND

A further subject of the present disclosure is a method for reductively decoloring dyed keratin fibers, particularly human hair, wherein the previously described multi-component packaging unit is used.

Preparations for tinting and dyeing hair are an important type of cosmetic agent. Said preparations can be used to nuance the natural hair color slightly or more heavily in accordance with the wishes of the particular person, to achieve a completely different hair color, or to conceal undesired shades of color, such as shades of gray. Depending on the desired color and/or lastingness of the coloring, typical hair dyeing agents are formulated either on the basis of oxidation dyes or on the basis of substantive dyes. Combinations of oxidation dyes and substantive dyes are also frequently used to achieve specific nuances.

Dyeing agents based on oxidation dyes lead to brilliant and lasting shades of color. However, said dyeing agents require the use of strong oxidants, such as hydrogen peroxide. Such dyes contain oxidation dye intermediates: developer components and coupler components. The developer components form the actual dyes among each other or by coupling with one or more coupler components, under the influence of oxidants or atmospheric oxygen.

Dyeing agents based on substantive dyes are often used for temporary coloring. Substantive dyes are dye molecules that attach directly to the hair and do not require an oxidative process to form the color. Important representatives of this dye class are, for example, triphenylmethane dyes, azo dyes, anthraquinone dyes, or nitrobenzene dyes, each of which can also bear cationic or anionic groups.

In all these dyeing processes, it may happen that the coloring should be completely or partially reversed for various reasons. Partial removal of the coloring is useful, for example, if the dyeing result on the fibers turns out darker than desired. On the other hand, complete removal of the coloring can also be desired in some cases. For example, it is conceivable that hair should be dyed or tinted in a certain nuance for a specific occasion and then the original color should be recovered after a few days.

Various agents and methods for color removal are already known in the literature. A method for reversing colorings that is well known from the prior art is the oxidative decoloring of dyed hair, for example by employing a typical bleaching agent. However, in this process the fibers can be damaged by the use of strong oxidants.

Furthermore, reductive processes for color removal have also already been described. For example, the European patent application EP 1 300 136 A2 discloses a method for treating hair in which the hair is dyed in a first step and reductively decolored in a second step. In said method, the reductive decoloring is performed by using a formulation containing a dithionite salt and containing a surfactant. In WO 2008/055756 A2, the reductive decoloring of keratin fibers is performed by employing a mixture of a reductant and an absorbent.

In documents WO 2012/069599, WO 2014/174230, and WO 2013/017862, various sulfinic acid derivatives in agents for reductively removing color from dyed hair are described.

If reductive decoloring agents are used, the decoloring occurs by reduction of the dyes present on the keratin fibers or hair. As a result of the reduction, the dyes are generally converted into the reduced leuco forms thereof. In this process, the double bonds present in the dyes are reduced, the chromophoric system of the dyes is interrupted in this way, and the dye is converted into a colorless form.

A general problem of the reductive decoloring agents known from the prior art is that the dyed keratin fibers can initially be decolored by using the reductant but the color removal does not last. Particularly in the case of oxidatively dyed hair, in the case of which the coloring is produced on the hair by oxidation dye intermediates of the developer type and of the coupler type, colorings having very good fastness properties in some cases are obtained. When the reductive decoloring agent is used, these dyes are then reductively converted into colorless compounds—which, however, still remain on the hair due to similarly good fastness properties.

After the reductant has been rinsed off, these reduced forms can then be gradually oxidized again under the influence of atmospheric oxygen. Because of this reoxidation, more or less pronounced recoloring occurs. This recoloring generally does not correspond to the shade of color in which the keratin fibers had been previously dyed, but rather can turn out unattractive in any manner and is therefore desired by the user of the decoloring agent all the less.

The problem addressed by the present disclosure is that of providing a decoloring agent for decoloring dyed keratin fibers that decolors dyed keratin fibers as completely as possible. The decoloring should be long-lasting, and the decolored keratin fibers should not suffer any recoloring, any nuance shift, or any post-darkening under the influence of atmospheric oxygen. The decoloring agent should exhibit good decoloring performance especially on keratin fibers that have been previously dyed by employing oxidative dyeing agents based on oxidation dye intermediates of the developer type and of the coupler type.

### BRIEF SUMMARY

Multi-component packaging units for reductively decoloring dyed keratin fibers and methods for reductively decoloring dyed keratin fibers are provided herein. In an embodiment, a multi-component packaging unit for reductively decoloring dyed keratin fibers includes a container (A) that includes a cosmetic agent (a) and a container (B) that includes a cosmetic agent (b), which are produced separately from each other. Agent (a) in container (A) includes one or more sulfur-containing reductants. Agent (b) in container (B) includes one or more metal salts chosen from the group of tin(II) salts, iron(II) salts, manganese(II) salts, titanium(II) salts, or titanium(III) salts.

In another embodiment, a method for reductively decoloring dyed keratin fibers includes the following steps in the indicated order:

(I) producing a ready-to-use decoloring agent by mixing an agent (a) that includes one or more sulfur-containing reductants, with a cosmetic agent (c) having a water content of from about 40.0 to about 99.0 wt % and comprising one or more acids or one or more alkalizing agents,

(II) applying the ready-to-use decoloring agent to keratin fibers,

(III) allowing the decoloring agent to act,

(IV) optionally rinsing the decoloring agent off of the keratin fibers,

(V) applying a cosmetic agent (b) to the keratin fibers, wherein agent (b) comprises one or more metal salts chosen from the group of tin(II) salts, iron(II) salts, manganese(II) salts, titanium(II) salts, or titanium(III) salts,

(VI) allowing agent (b) to act, and

(VII) optionally rinsing agent (b) off of the keratin fibers.

In another embodiment, a multi-component packaging unit for reductively decoloring dyed keratin fibers is provided. The multi-component packaging unit includes a container (A) including a cosmetic agent (a), a container (B) including a cosmetic agent (b), and a container (C) containing a cosmetic agent (c), which are produced separately from each other. Agent (a) in container (A) includes one or more sulfur-containing reductants chosen from the group of (H₂N)(NH)C(SO₂H) formamidine sulfinic acid, sodium dithionite, zinc dithionite, N(CH₂SO₂Na)₃ trisodium [bis(sulfinatomethyl)amino]methanesulfinate, N(CH₂SO₂K)₃ tripotassium [bis(sulfinatomethyl)amino]methanesulfinate, or N(CH₂SO₂H)₃ [bis(sulfinomethyl)amino]methanesulfinic acid, wherein the one or more sulfur-containing reductants are present in a total amount of from about 35.0 to about 55.0 wt %, with respect to the total weight of agent (a). Agent (a) in container (A) further includes one or more fatty constituents present in a total amount of from about 5.0 to about 15.0 wt %, with respect to the total weight of agent (a). Agent (a) in container (A) has a water content of at most about 1.0 wt %, with respect to the total weight of agent (a). Agent (b) in container (B) includes one or more metal salts chosen from the group of tin(II) chloride, tin(II) bromide, tin(II) fluoride, tin(II) acetate, tin(II) gluconate, tin(II) lactate, or tin(II) tartrate, wherein the one or more metal salts are present in a total amount of from about 3.0 to about 10.0 wt %, with respect to the total weight of agent (b). Agent (b) in container (B) has a water content of from about 70 to about 93.0 wt %, with respect to the total weight of agent (b). Agent (c) in container (C) includes one or more acids or one or more alkalizing agents. Agent (c) has a water content of from about 70 to about 99.0 wt %, with respect to the total weight of agent (c).

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has now been found that this problem can be solved by using a multi-component packaging unit that comprises two separately produced containers. The first container (A) contains a first agent (a), which contains one or more sulfur-containing reductants. The second, separately produced container (B) contains a second cosmetic agent (b), which contains one or more specific, reductive metal salts. Surprisingly, by post-treatment of the fibers first reductively decolored by (a) with the metal salts (b), it was possible to further improve the decoloring result and to effectively inhibit the reoxidation of the dyes (i.e., the post-darkening under the influence of atmospheric oxygen).

A first subject of the present disclosure is a multi-component packaging unit (kit of parts) for reductively decoloring dyed keratin fibers, particularly human hair, which comprises

a container (A) containing a cosmetic agent (a) and

a container (B) containing a cosmetic agent (b),

which are produced separately from each other, wherein

agent (a) in container (A) contains one or more sulfur-containing reductants and

agent (b) in container (B) contains one or more metal salts from the group of the tin(II) salts, the iron(II) salts, the manganese(II) salts, the titanium(II) salts, and the titanium(III) salts.

The terms “keratin fibers” and “keratin-containing fibers” should be understood to mean pelts, wool, feathers, and, in particular, human hair. Although the agents as contemplated herein are suitable predominantly for lightening and coloring keratin fibers or human hair, there is, in principle, nothing standing in the way of use in other fields.

The term “dyed keratin fibers” is understood to mean keratin fibers that have been dyed by employing traditional cosmetic dyeing agents known to a person skilled in the art. In particular, the term “dyed keratin fibers” should be understood to mean fibers that have been dyed by employing the oxidative dyeing agents known from the prior art and/or by employing substantive dyes. In this context, the known monographs, e.g., Kh. Schrader, “Grundlagen and Rezepturen der Kosmetika”, 2nd edition, Hüthig Buch Verlag, Heidelberg, 1989, that represent the relevant knowledge of a person skilled in the art are expressly referenced.

The multi-component packaging unit as contemplated herein comprises the separately produced containers (A) and (B), wherein container (A) contains agent (a) and container (B) contains agent (b). Agents (a) and (b) should be used successively on the dyed keratin fibers.

Agent (a) contains one or more sulfur-containing reductants, which are responsible for reductively decoloring the artificial dyes present on the keratin fiber in a first step.

After agent (a) has been applied to the fibers, agent (a) is preferably left there for a time span of from about 30 seconds to about 120 minutes. Optionally, agent (a) can be rinsed from the fibers before agent (b) is applied to the fibers. After agent (b) has been applied to the fibers, agent (b) also is preferably left there for a time span of from about 30 seconds to about 120 minutes. Optionally, agent (b) can thereafter be rinsed from the fibers.

Agent (a) in Container (A)

The multi-component packaging unit (kit of parts) as contemplated herein comprises a first container (A), which contains a cosmetic agent (a). Agent (a) is characterized by the content of one or more sulfur-containing reductants therein.

The one or more reductants are preferably selected from the group consisting of (H₂N)(NH)C(SO₂H) formamidine sulfinic acid, sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, thioglycolic acid, HN(CH₂SO₂Na)₂ disodium [(sulfinatomethyl)amino]methanesulfinate, HN(CH₂SO₂K)₂ dipotassium [(sulfinatomethyl)amino]methanesulfinate, HN(CH₂SO₂H)₂ [(sulfinomethyl)amino]methanesulfinic acid, N(CH₂SO₂Na)₃ trisodium [bis(sulfinatomethyl)amino]methanesulfinate, N(CH₂SO₂K)₃ tripotassium [bis(sulfinatomethyl)amino]methanesulfinate, N(CH₂SO₂H)₃ [bis(sulfinomethyl)amino]methanesulfinic acid, H₂NCH(CH₃)SO₂Na sodium 1-aminoethane-1-sulfinate, H₂NCH(CH₃)SO₂K potassium 1-aminoethane-1-sulfinate, H₂NCH(CH₃)SO₂H 1-aminoethane-1-sulfinic acid, HN(CH(CH₃)SO₂Na)₂ disodium 1-[(1-sulfinatoethyl)amino]ethane-1-sulfinate, HN(CH(CH₃)SO₂K)₂ dipotassium 1-[(1-sulfinatoethyl)amino]ethane-1-sulfinate, HN(CH(CH₃)SO₂H)₂ 1-[(1-sulfinoethyl)amino]ethane-1-sulfinic acid, N(CH(CH₃)SO₂Na)₃ trisodium 1-[bis(1-sulfinatoethyl)amino]ethane-1-sulfinate, N(CH(CH₃)SO₂K)₃ tripotassium 1-[bis(1-sulfinatoethyl)amino]ethane-1-sulfinate, and N(CH(CH₃)SO₂H)₃ 1-[bis(1-sulfinoethyl)amino]ethane-1-sulfinic acid.

In an exceedingly preferred embodiment, the multi-component packaging unit (kit of parts) as contemplated herein is provided wherein agent (a) in container (A) contains one or more sulfur-containing reductants from the group consisting of (H₂N)(NH)C(SO₂H) formamidine sulfinic acid, sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, thioglycolic acid, HN(CH₂SO₂Na)₂ disodium [(sulfinatomethyl)amino]methanesulfinate, HN(CH₂SO₂K)₂ dipotassium [(sulfinatomethyl)amino]methanesulfinate, HN(CH₂SO₂H)₂ [(sulfinomethyl)amino]methanesulfinic acid, N(CH₂SO2Na)₃ trisodium [bis(sulfinatomethyl)amino]methanesulfinate, N(CH₂SO₂K)₃ tripotassium [bis(sulfinatomethyl)amino]methanesulfinate, N(CH₂SO₂H)₃ [bis(sulfinomethyl)amino]methanesulfinic acid, H₂NCH(CH₃)SO₂Na sodium 1-aminoethane-1-sulfinate, H₂NCH(CH₃)SO₂K potassium 1-aminoethane-1-sulfinate, H₂NCH(CH₃)SO₂H 1-aminoethane-1-sulfinic acid, HN(CH(CH₃)SO₂Na)₂ disodium 1-[(1-sulfinatoethyl)amino]ethane-1-sulfinate, HN(CH(CH₃)SO₂K)₂ dipotassium 1-[(1-sulfinatoethyl)amino]ethane-1-sulfinate, HN(CH(CH₃)SO₂H)₂ 1-[(1-sulfinoethyl)amino]ethane-1-sulfinic acid, N(CH(CH₃)SO₂Na)₃ trisodium 1-[bis(1-sulfinatoethyl)amino]ethane-1-sulfinate, N(CH(CH₃)SO₂K)₃ tripotassium 1-[bis(1-sulfinatoethyl)amino]ethane-1-sulfinate, and N(CH(CH₃)SO₂H)₃ 1-[bis(1-sulfinoethyl)amino]ethane-1-sulfinic acid.

Formamidine sulfinic acid is alternatively also called thiourea dioxide or aminoiminomethanesulfinic acid. Formamidine sulfinic acid has the structure of formula (I) but can also be present in the form of the tautomers of formamidine sulfinic acid. Formamidine sulfinic acid has the CAS number 1758-73-2 and is commercially available from various suppliers, such as Sigma Aldrich.

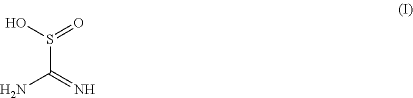

Sodium dithionite is an inorganic reductant having the empirical formula Na₂S₂O₄ and the CAS no. 7775-14-6.

Zinc dithionite is an inorganic reductant having the empirical formula ZnS₂O₄ and the CAS no. 7779-86-4.

Potassium dithionite is an inorganic reductant having the empirical formula K₂S₂O₄ and the CAS no. 14293-73-3.

Sodium sulfite is an inorganic reductant having the empirical formula Na2SO3 and the CAS no. 7757-83-7.

Sodium hydrogen sulfite is an inorganic reductant having the empirical formula NaHSO3 and the CAS no. 7631-90-5. Sodium hydrogen sulfite is preferably used in the form of an aqueous solution.

Potassium sulfite is an inorganic reductant having the empirical formula K2SO3 and the CAS no. 10117-38-1.

Potassium hydrogen sulfite is an inorganic reductant having the empirical formula KHSO3 and the CAS no. 7773-03-7.

Ammonium sulfite is an inorganic reductant having the empirical formula (NH4)2SO3 and the CAS no. 10196-04-0.

Sodium thiosulfate is an inorganic reductant having the empirical formula Na2S2O3 and the CAS no. 7772-98-7.

Potassium thiosulfate is an inorganic reductant having the empirical formula K2S2O3 and the CAS no. 10294-66-3.

Ammonium thiosulfate is an inorganic reductant having the empirical formula (NH4)2S2O3 and the CAS no. 7783-18-8.

Hydroxymethanesulfinic acid is an organic reductant having the formula HO—CH2-S(O)OH and the CAS no. 79-25-4. Hydroxymethanesulfinic acid is alternatively also called formaldehydesulfoxylic acid. Both the use of hydroxymethanesulfinic acid itself and the use of the physiologically acceptable salts of hydroxymethanesulfinic acid, for example the sodium salt and/or the zinc salt, are contemplated herein. The use of sodium formaldehyde sulfoxylate (sodium hydroxymethanesulfinate, the sodium salt of hydroxymethanesulfinic acid) and/or zinc formaldehyde sulfoxylate (zinc hydroxymethanesulfinate, the zinc salt of hydroxymethanesulfinic acid) is therefore likewise contemplated herein.

Aminomethanesulfinic acid is an organic reductant having the formula H2N—CH2-S(O)OH and the CAS no. 118201-33-5. Both the use of aminomethanesulfinic acid itself and the use of the physiologically acceptable salts of aminomethanesulfinic acid, for example the sodium salt and/or the zinc salt, are contemplated herein. The use of sodium aminomethane sulfinate (the sodium salt of aminomethanesulfinic acid) and/or zinc aminomethane sulfinate (the zinc salt of aminomethanesulfinic acid) is therefore likewise contemplated herein.

As contemplated herein, cysteine (2-amino-3-sulfanylpropanoic acid) is understood to mean D-cysteine, L-cysteine, and/or a mixture of D- and L-cysteine.

Thiolactic acid (2-sulfanylpropanoic acid) is understood to mean D-thiolactic acid, L-thiolactic acid, and/or a mixture of D- and L-thiolactic acid. Both the use of thiolactic acid itself and the use of thiolactic acid in the form of a physiologically acceptable salt thereof are contemplated herein. A preferred salt of thiolactic acid is ammonium thiolactate.

Ammonium thiolactate is the ammonium salt of thiolactic acid (i.e., the ammonium salt of 2-sulfanylpropanoic acid) (formula II).

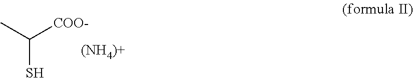

The definition of ammonium thiolactate also comprises the ammonium salts of D-thiolactic acid and the ammonium salts of L-thiolactic acid, and mixtures thereof.

Thioglycolic acid (sulfanylacetic acid, 2-mercaptoacetic acid) is understood to mean an organic reductant of the formula HS—CH₂—COOH. The compound has the CAS no. 68-11-1. In the case of thioglycolic acid as well, both the use of thioglycolic acid itself and the use of a physiologically acceptable salt of thioglycolic acid are contemplated herein. For example, sodium thioglycolate, potassium thioglycolate, and/or ammonium thioglycolate can be used as physiologically acceptable salts of thioglycolic acid. Ammonium thioglycolate is a preferred physiologically acceptable salt of thioglycolic acid.

Ammonium thioglycolate is the ammonium salt of thioglycolic acid (i.e., the ammonium salt of sulfanylacetic acid) (formula III).

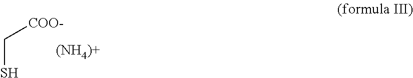

Disodium [(sulfinatomethyl)amino]methanesulfinate is the disodium salt of [(sulfinomethyl)amino]methanesulfinic acid and has the structure of formula (IV).

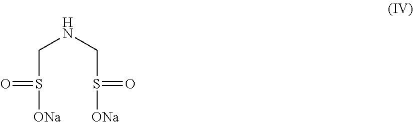

Dipotassium [(sulfinatomethyl)amino]methanesulfinate is the dipotassium salt of [(sulfinomethyl)amino]methanesulfinic acid and has the structure of formula (V).

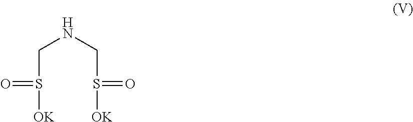

[(Sulfinomethyl)amino]methanesulfinic acid has the structure of formula (VI).

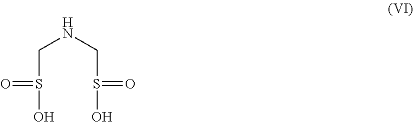

Trisodium [bis(sulfinatomethyl)amino]methanesulfinate is the trisodium salt of [bis(sulfinomethyl)amino]methanesulfinic acid and has the structure of formula (VII).

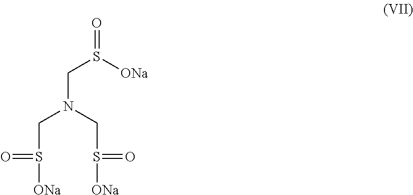

Tripotassium [bis(sulfinatomethyl)amino]methanesulfinate is the tripotassium salt of [bis(sulfinomethyl)amino]methanesulfinic acid and has the structure of formula (VIII).

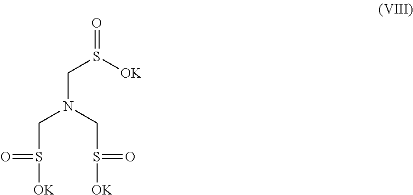

[Bis(sulfinomethyl)amino]methanesulfinic acid has the structure of formula (IX).

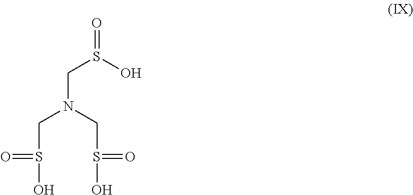 [SMILES: O=S(O)CN(CS(=O)O)CS(=O)O]

Sodium 1-aminoethane-1-sulfinate is the sodium salt of 1-aminoethane-1-sulfinic acid and has the structure of formula (X).

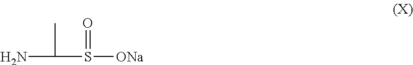

Potassium 1-aminoethane-1-sulfinate is the potassium salt of 1-aminoethane-1-sulfinic acid and has the structure of formula (XI).

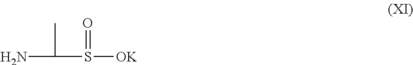

1-Aminoethane-1-sulfinic acid has the structure of formula (XII).

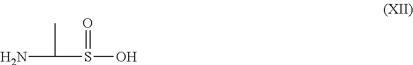

Disodium 1-[(1-sulfinatoethyl)amino]ethane-1-sulfinate is the disodium salt of 1-[(1-sulfinoethyl)amino]ethane-1-sulfinic acid and has the structure of formula (XIII).

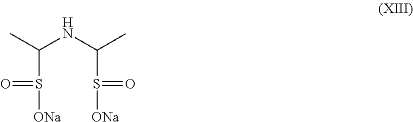

Dipotassium 1-[(1-sulfinatoethyl)amino]ethane-1-sulfinate is the dipotassium salt of 1-[(1-sulfinoethyl)amino]ethane-1-sulfinic acid and has the structure of formula (XIV).

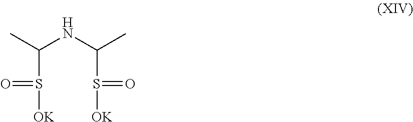

1-[(1-Sulfinoethyl)amino]ethane-1-sulfinic acid has the structure of formula (XV).

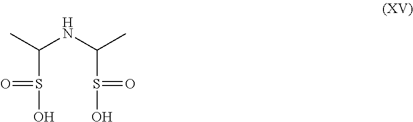

Trisodium 1-[bis(1-sulfinatoethyl)amino]ethane-1-sulfinate is the trisodium salt of 1-[bis(1-sulfinoethyl)amino]ethane-1-sulfinic acid and has the structure of formula (XVI).

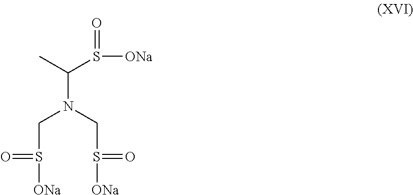

Tripotassium 1-[bis(1-sulfinatoethyl)amino]ethane-1-sulfinate is the tripotassium salt of 1-[bis(1-sulfinoethyl)amino]ethane-1-sulfinic acid and has the structure of formula (XVII).

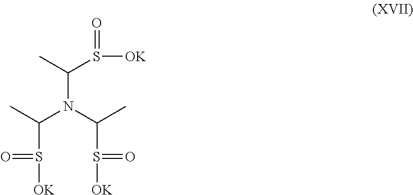

1-[Bis(1-sulfinoethyl)amino]ethane-1-sulfinic acid has the structure of formula (XVIII).

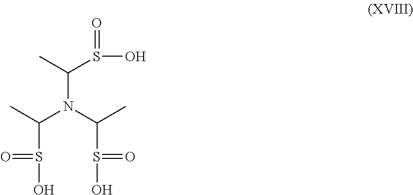 [SMILES: CC(N(C(C)S(=O)O)C(C)S(=O)O)S(=O)O]

The production of the compounds of formulas (IV) to (XVIII) is described in EP 0914516 B1, for example.

It has been found to be exceedingly preferred if one or more compounds from the group consisting of (H₂N)(NH)C(SO₂H) formamidine sulfinic acid, sodium dithionite, zinc dithionite, N(CH₂SO₂Na)₃ trisodium [bis(sulfinatomethyl)amino]methanesulfinate, N(CH₂SO₂K)₃ tripotassium [bis(sulfinatomethyl)amino]methanesulfinate, and N(CH₂SO₂H)₃ [bis(sulfinomethyl)amino]methanesulfinic acid are used as sulfur-containing reductants in agent (a).

In an especially preferred embodiment, a multi-component packaging unit (kit of parts) as contemplated herein is therefore provided wherein agent (a) in container (A) contains one or more sulfur-containing reductants from the group consisting of (H₂N)(NH)C(SO₂H) formamidine sulfinic acid, sodium dithionite, zinc dithionite, N(CH₂SO₂Na)₃ trisodium [bis(sulfinatomethyl)amino]methanesulfinate, N(CH₂SO₂K)₃ tripotassium [bis(sulfinatomethyl)amino]methanesulfinate, and N(CH₂SO₂H)₃ [bis(sulfinomethyl)amino]methanesulfinic acid.

Furthermore, the one or more sulfur-containing reductants are preferably used in certain amount ranges. Agent (a) preferably contains the one or more sulfur-containing reductants in a total amount of from about 0.1 to about 80.0 wt %, from about 5.0 to about 75.0 wt %, from about 15.0 to about 65.0 wt %, or from about 35.0 to about 55.0 wt %, with respect to the total weight of agent (a).

In a further, especially preferred embodiment, a multi-component packaging unit (kit of parts) as contemplated herein is therefore provided wherein agent (a) in container (A) contains one or more sulfur-containing reductants in a total amount of from about 0.1 to about 80.0 wt %, from about 5.0 to about 75.0 wt %, from about 15.0 to about 65.0 wt %, or from about 35.0 to about 55.0 wt %, with respect to the total weight of agent (a).

Exceedingly preferred is a multi-component packaging unit (kit of parts) for reductively decoloring dyed keratin fibers that is provided wherein agent (a) in container (A) contains
(a1) from about 0.1 to about 80.0 wt % of formamidine sulfinic acid (alternative name: thiourea dioxide).

Exceedingly preferred is a multi-component packaging unit (kit of parts) for reductively decoloring dyed keratin fibers that is provided wherein agent (a) in container (A) contains
(a1) from about 5.0 to about 75.0 wt % of formamidine sulfinic acid (alternative name: thiourea dioxide).

Exceedingly preferred is a multi-component packaging unit (kit of parts) for reductively decoloring dyed keratin fibers that is provided wherein agent (a) in container (A) contains
(a1) from about 15.0 to about 65.0 wt % of formamidine sulfinic acid (alternative name: thiourea dioxide).

Exceedingly preferred is a multi-component packaging unit (kit of parts) for reductively decoloring dyed keratin fibers that is provided wherein agent (a) in container (A) contains
(a1) from about 35.0 to about 55.0 wt % of formamidine sulfinic acid (alternative name: thiourea dioxide).

Exceedingly preferred is a multi-component packaging unit (kit of parts) for reductively decoloring dyed keratin fibers that is provided wherein agent (a) in container (A) contains
(a1) from about 0.1 to about 80.0 wt % of sodium dithionite.

Exceedingly preferred is a multi-component packaging unit (kit of parts) for reductively decoloring dyed keratin fibers that is provided wherein agent (a) in container (A) contains
(a1) from about 5.0 to about 75.0 wt % of sodium dithionite.

Exceedingly preferred is a multi-component packaging unit (kit of parts) for reductively decoloring dyed keratin fibers that is provided wherein agent (a) in container (A) contains
(a1) from about 15.0 to about 65.0 wt % of sodium dithionite.

Exceedingly preferred is a multi-component packaging unit (kit of parts) for reductively decoloring dyed keratin fibers that is provided wherein agent (a) in container (A) contains
(a1) from about 35.0 to about 55.0 wt % of sodium dithionite.

Exceedingly preferred is a multi-component packaging unit (kit of parts) for reductively decoloring dyed keratin fibers that is provided wherein agent (a) in container (A) contains
(a1) from about 0.1 to about 80.0 wt % of zinc dithionite.

Exceedingly preferred is a multi-component packaging unit (kit of parts) for reductively decoloring dyed keratin fibers that is provided wherein agent (a) in container (A) contains
(a1) from about 5.0 to about 75.0 wt % of zinc dithionite.

Exceedingly preferred is a multi-component packaging unit (kit of parts) for reductively decoloring dyed keratin fibers that is provided wherein agent (a) in container (A) contains
(a1) from about 15.0 to about 65.0 wt % of zinc dithionite.

Exceedingly preferred is a multi-component packaging unit (kit of parts) for reductively decoloring dyed keratin fibers that is provided wherein agent (a) in container (A) contains
(a1) from about 35.0 to about 55.0 wt % of zinc dithionite.

Exceedingly preferred is a multi-component packaging unit (kit of parts) for reductively decoloring dyed keratin fibers that is provided wherein agent (a) in container (A) contains
(a1) from about 0.1 to about 80.0 wt % of trisodium [bis(sulfinatomethyl)amino]methanesulfinate.

Exceedingly preferred is a multi-component packaging unit (kit of parts) for reductively decoloring dyed keratin fibers that is provided wherein agent (a) in container (A) contains
(a1) from about 5.0 to about 75.0 wt % of trisodium [bis(sulfinatomethyl)amino]methanesulfinate.

Exceedingly preferred is a multi-component packaging unit (kit of parts) for reductively decoloring dyed keratin fibers that is provided wherein agent (a) in container (A) contains
(a1) from about 15.0 to about 65.0 wt % of trisodium [bis(sulfinatomethyl)amino]methanesulfinate.

Exceedingly preferred is a multi-component packaging unit (kit of parts) for reductively decoloring dyed keratin fibers that is provided wherein agent (a) in container (A) contains
(a1) from about 35.0 to about 55.0 wt % of trisodium [bis(sulfinatomethyl)amino]methanesulfinate.

Exceedingly preferred is a multi-component packaging unit (kit of parts) for reductively decoloring dyed keratin fibers that is provided wherein agent (a) in container (A) contains
(a1) from about 0.1 to about 80.0 wt % of tripotassium [bis(sulfinatomethyl)amino]methanesulfinate.

Exceedingly preferred is a multi-component packaging unit (kit of parts) for reductively decoloring dyed keratin fibers that is provided wherein agent (a) in container (A) contains
(a1) from about 5.0 to about 75.0 wt % of tripotassium [bis(sulfinatomethyl)amino]methanesulfinate.

Exceedingly preferred is a multi-component packaging unit (kit of parts) for reductively decoloring dyed keratin fibers that is provided wherein agent (a) in container (A) contains
(a1) from about 15.0 to about 65.0 wt % of tripotassium [bis(sulfinatomethyl)amino]methanesulfinate.

Exceedingly preferred is a multi-component packaging unit (kit of parts) for reductively decoloring dyed keratin fibers that is provided wherein agent (a) in container (A) contains
(a1) from about 35.0 to about 55.0 wt % of tripotassium [bis(sulfinatomethyl)amino]methanesulfinate.

Exceedingly preferred is a multi-component packaging unit (kit of parts) for reductively decoloring dyed keratin fibers that is provided wherein agent (a) in container (A) contains
(a1) from about 0.1 to about 80.0 wt % of [bis(sulfinomethyl)amino]methanesulfinic acid.

Exceedingly preferred is a multi-component packaging unit (kit of parts) for reductively decoloring dyed keratin fibers that is provided wherein agent (a) in container (A) contains
(a1) from about 5.0 to about 75.0 wt % of [bis(sulfinomethyl)amino]methanesulfinic acid.

Exceedingly preferred is a multi-component packaging unit (kit of parts) for reductively decoloring dyed keratin fibers that is provided wherein agent (a) in container (A) contains
(a1) from about 15.0 to about 65.0 wt % of [bis(sulfinomethyl)amino]methanesulfinic acid.

Exceedingly preferred is a multi-component packaging unit (kit of parts) for reductively decoloring dyed keratin fibers that is provided wherein agent (a) in container (A) contains
(a1) from about 35.0 to about 55.0 wt % of [bis(sulfinomethyl)amino]methanesulfinic acid.

Agent (a) contains the one or more sulfur-containing reductants essential to the invention in a cosmetic carrier, which can be formulated to contain water or to be substantially free of water.

Exceedingly preferably, agent (a) is formulated to be substantially free of water and can exist in solid form, as a powder, or as a paste. The formulation of agent (a) as a paste is preferred.

Agent (a) can additionally contain one or more fatty constituents. The one or more fatty constituents are preferably used in agent (a) in a total amount of from about 5.0 to about 45.0 wt %, from about 5.0 to about 35.0 wt %, from about 5.0 to about 25.0 wt %, or from about 5.0 to about 15.0 wt %, with respect to the total weight of agent (a).

In an especially preferred embodiment, a multi-component packaging unit (kit of parts) as contemplated herein is provided wherein agent (a) in container (A) contains one or more fatty constituents in a total amount from about of 5.0 to about 45.0 wt %, from about 5.0 to about 35.0 wt %, from about 5.0 to about 25.0 wt %, or from about 5.0 to about 15.0 wt %, with respect to the total weight of agent (a).

Furthermore, particularly if agent (a) is formulated to be substantially free of water, the chosen content of the fatty constituents from the group in agent (a) can be so high that the fatty constituents act as a carrier of agent (a) and therefore—in addition to the sulfur-containing reductants—are the main constituent of agent (a).

As contemplated herein, the term “fatty constituents” is understood to mean organic compounds having a solubility in water at room temperature (22° C.) and atmospheric pressure (760 mm Hg) of less than from 1 wt %, such as less than about 0.1 wt %.

Explicitly only uncharged (i.e., non-ionic) compounds fall under the definition of “fatty constituents”. Fatty constituents have at least one saturated or unsaturated alkyl group having at least 12 C atoms. The molecular weight of the fatty constituents is at most about 5000 g/mol, preferably at most about 2500 g/mol, and especially preferably at most about 1000 g/mol. The fatty constituents are neither polyoxyalkylated compounds nor polyglycerylated compounds. In this context, polyalkoxylated compounds are compounds in the production of which at least 2 alkylene oxide units were reacted. Similarly, polyglycerated compounds are compounds in the production of which at least two glycerol units were converted.

Because explicitly only non-ionic substances are considered to be fatty constituents in the sense of the present disclosure, charged compounds, such as fatty acids and the salts of fatty acids, do not fall within the group of the fatty constituents.

Preferred fatty constituents are the constituents from the group of the C₁₂-C₃₀ fatty alcohols, the C₁₂-C₃₀ fatty acid triglycerides, the C₁₂-C₃₀ fatty acid monoglycerides, the C₁₂-C₃₀ fatty acid diglycerides, the C₁₂-C₃₀ fatty acid esters, and/or the hydrocarbons.

The C12-C30 fatty alcohols can be saturated, mono- or polyunsaturated, linear or branched fatty alcohols having 12 to 30 C atoms.

Examples of preferred linear, saturated C12-C30 fatty alcohols are dodecan-1-ol (dodecyl alcohol, lauryl alcohol), tetradecan-1-ol (tetradecyl alcohol, myristyl alcohol), hexadecan-1-ol (hexadecyl alcohol, cetyl alcohol, palmityl alcohol), octadecan-1-ol (octadecyl alcohol, stearyl alcohol), arachidyl alcohol (icosan-1-ol), henicosyl alcohol (henicosan-1-ol), and/or behenyl alcohol (docosan-1-ol).

Preferred linear, unsaturated fatty alcohols are (9Z)-octadec-9-en-1-ol (oleyl alcohol), (9E)-octadec-9-en-1-ol (elaidyl alcohol), (9Z,12Z)-octadeca-9,12-dien-1-ol (linoleyl alcohol), (9Z,12Z,15Z)-octadeca-9,12,15-trien-1-ol (linolenyl alcohol), gadoleyl alcohol ((9Z)-icos-9-en-1-ol), arachidonyl alcohol ((5Z,8Z,11Z,14Z)-icosa-5,8,11,14-tetraen-1-ol), erucyl alcohol ((13Z)-docos-13-en-1-ol), and/or brassidyl alcohol ((13E)-docosen-1-ol).

The preferred representatives of branched fatty alcohols are 2-octyldodecanol, 2-hexyldodecanol, and/or 2-butyldodecanol.

In the sense of the present disclosure, the term “C12-C30 fatty acid triglyceride” is understood to mean the triester of the trihydric alcohol glycerol with three equivalents of fatty acid. Structurally identical or different fatty acids can be involved in the esterification within a triglyceride molecule.

As contemplated herein, the term “fatty acids” should be understood to mean saturated or unsaturated, unbranched or branched, unsubstituted or substituted C12-C30 carboxylic acids. Unsaturated fatty acids can be mono- or polyunsaturated. The C—C double bond(s) of an unsaturated fatty acid can have the cis or trans configuration.

Especially suitable are the fatty acid triglycerides in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), icosanoic acid (arachidic acid), docosanoic acid (behenic acid), petroselic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid], linolenic acid [(9Z,12Z,15Z)-octadeca-9,12,15-trienoic acid], elaeostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid].

The fatty acid triglycerides can also be of natural origin. The fatty acid triglycerides occurring in soy oil, peanut oil, olive oil, sunflower oil, macadamia nut oil, moringa oil, apricot kernel oil, manila oil, and/or optionally hardened castor oil and mixtures thereof are especially suitable for use in agent (a).

The term “C12-C30 fatty acid monoglyceride” is understood to mean the monoester of the trihydric alcohol glycerol with an equivalent of fatty acid. Either the central hydroxy group of the glycerol or the terminal hydroxy group of the glycerol can be esterified with the fatty acid.

Especially suitable are the C12-C30 fatty acid monoglycerides in which a hydroxy group of the glycerol is esterified with a fatty acid, wherein the fatty acids are selected from dodecanoic acid (lauric acid), tetradecanoic acid (myristic acid), hexadecanoic acid (palmitic acid), tetracosanoic acid (lignoceric acid), octadecanoic acid (stearic acid), icosanoic acid (arachidic acid), docosanoic acid (behenic acid), petroselic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid], linolenic acid [(9Z,12Z,15Z)-octadeca-9,12,15-trienoic acid], elaeostearic acid [(9Z,11E,13E)-octadeca-9,11,3-trienoic acid], arachidonic acid [(5Z,8Z,11Z,14Z)-icosa-5,8,11,14-tetraenoic acid], or nervonic acid [(15Z)-tetracos-15-enoic acid].

The term “C₁₂-C₃₀ fatty acid diglyceride” is understood to mean the diester of the trihydric alcohol glycerol with two equivalents of fatty acid. Either the central hydroxy group and one terminal hydroxy group of the glycerol can be esterified with two equivalents of fatty acid or the two terminal hydroxy groups of the glycerol can be esterified with one fatty acid each. The glycerol can be esterified with two structurally identical fatty acids or two different fatty acids.

Especially suitable are the fatty acid diglycerides in which at least one of the ester groups is formed from glycerol with a fatty acid selected from dodecanoic acid (lauric acid), tetradecanoic acid (myristic acid), hexadecanoic acid (palmitic acid), tetracosanoic acid (lignoceric acid), octadecanoic acid (stearic acid), icosanoic acid (arachidic acid), docosanoic acid (behenic acid), petroselic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid], linolenic acid [(9Z,12Z,15Z)-octadeca-9,12,15-trienoic acid], elaeostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid].

In the sense of the present disclosure, the term “C12-C30 fatty acid ester” is understood to mean the monoester of a fatty acid and an aliphatic, monohydric alcohol, wherein the alcohol comprises up to 6 C atoms. Ethanol, n-propanol, isopropanol, 1-butanol, isobutanol, tert-butanol, n-pentanol, isopentanol, and n-hexanol, for example, can be named as suitable alcohols. Preferred alcohols are ethanol and isopropanol.

Preferred C12-C30 fatty acid esters are the esters that are formed when the alcohols ethanol and/or isopropanol are esterified with one of the fatty acids from the group of dodecanoic acid (lauric acid), tetradecanoic acid (myristic acid), hexadecanoic acid (palmitic acid), tetracosanoic acid (lignoceric acid), octadecanoic acid (stearic acid), icosanoic acid (arachidic acid), docosanoic acid (behenic acid), petroselic acid [(Z)-6-octadecenoic acid], palmitoleic acid [(9Z)-hexadec-9-enoic acid], oleic acid [(9Z)-octadec-9-enoic acid], elaidic acid [(9E)-octadec-9-enoic acid], erucic acid [(13Z)-docos-13-enoic acid], linoleic acid [(9Z, 12Z)-octadeca-9,12-dienoic acid], linolenic acid [(9Z,12Z,15Z)-octadeca-9,12,15-trienoic acid], elaeostearic acid [(9Z,11E,13E)-octadeca-9,11,3-trienoic acid], arachidonic acid [(5Z,8Z,11Z,14Z)-icosa-5,8,11,14-tetraenoic acid], and/or nervonic acid [(15Z)-tetracos-15-enoic acid]. Among the fatty acid esters, isopropyl myristate is exceedingly preferred.

Hydrocarbons are mixtures of compounds having 8 to 250 C atoms, preferably having 12 to 150 C atoms, said mixtures consisting exclusively of the atoms carbon and hydrogen. In particular, aliphatic hydrocarbons such as mineral oils, liquid paraffin oils (e.g., paraffinum liquidum or paraffinum perliquidum), isoparaffin oils, semisolid paraffin oils, paraffin waxes, hard paraffin (paraffinum solidum), petroleum jelly, and polydecenes are preferred in this context.

Liquid paraffin oils (paraffinum liquidum or paraffinum perliquidum) have proven especially suitable in this context. The hydrocarbon is exceedingly preferably paraffinum liquidum, also called white oil. Paraffinum liquidum is a mixture of purified, saturated, aliphatic hydrocarbons that consists mainly of hydrocarbon chains having a C chain distribution of 25 to 35 C atoms.

An especially preferred multi-component packaging unit (kit of parts) for reductively decoloring dyed keratin fibers is provided wherein agent (a) in container (A) contains one or more fatty constituents from the group of the C₁₂-C₃₀ fatty alcohols in a total amount of from about 5.0 to about 45.0 wt %, from about 5.0 to about 35.0 wt %, from about 5.0 to about 25.0 wt %, or from about 5.0 to about 15.0 wt %, with respect to the total weight of agent (a).

In a further, preferred embodiment, a multi-component packaging unit (kit of parts) as contemplated herein is provided wherein agent (a) in container (A) contains one or more fatty constituents from the group of the C12-C30 fatty acid triglycerides in a total amount of from about 5.0 to about 45.0 wt %, from about 5.0 to about 35.0 wt %, from about 5.0 to about 25.0 wt %, or from about 5.0 to about 15.0 wt %, with respect to the total weight of agent (a).

In a further, preferred embodiment, a multi-component packaging unit (kit of parts) as contemplated herein is provided wherein agent (a) in container (A) contains one or more fatty constituents from the group of the C12-C30 fatty acid monoglycerides in a total amount of from about 5.0 to about 45.0 wt %, from about 5.0 to about 35.0 wt %, from about 5.0 to about 25.0 wt %, or from about 5.0 to about 15.0 wt %, with respect to the total weight of agent (a).

In a further, preferred embodiment, a multi-component packaging unit (kit of parts) as contemplated herein is provided wherein agent (a) in container (A) contains one or more fatty constituents from the group of the C12-C30 fatty acid diglycerides in a total amount of from about 5.0 to about 45.0 wt %, from about 5.0 to about 35.0 wt %, from about 5.0 to about 25.0 wt %, or from about 5.0 to about 15.0 wt %, with respect to the total weight of agent (a).

In a further, preferred embodiment, a multi-component packaging unit (kit of parts) as contemplated herein is provided wherein agent (a) in container (A) contains one or more fatty constituents from the group of the hydrocarbons in a total amount of from about 5.0 to about 70.0 wt %, from about 10.0 to about 65.0 wt %, from about 15.0 to about 60.0 wt %, or from about 25.0 to about 55.0 wt %, with respect to the total weight of agent (a).

Furthermore, agent (a) can also be formulated on the basis of a suitable aqueous or aqueous-alcoholic carrier. For the purpose of reductive decoloring, such carriers can be, for example, creams, emulsions, gels, or surfactant-containing foaming solutions, such as shampoos, foam aerosols, foam formulations, or other preparations suitable for use on hair. Suitable agents for reductively removing color from keratin fibers can be formulated as creams, emulsions, or flowable gels.

However, lowering the water content in agent (a) can prove advantageous for increasing the stability of agent (a), because in this way the one or more sulfur-containing reductants do not come in contact with water or come in contact with water only to a limited extend and therefore undesired reactions between water and reductant can be limited or avoided. In this context, it has proven preferable if agent (a) in container (A) has a water content of at most 10.0 wt %, preferably at most 5.0 wt %, more preferably at most 2.5 wt %, and exceedingly preferably at most 1.0 wt %, with respect to the total weight of agent (a).

In a further, preferred embodiment, a multi-component packaging unit (kit of parts) as contemplated herein is provided wherein agent (a) in container (A) has a water content of at most about 10.0 wt %, at most about 5.0 wt %, at most about 2.5 wt %, or at most about 1.0 wt %, with respect to the total weight of agent (a).

Even if agent (a) is formulated substantially water-free, a low water content can be desired in order, for example, to integrate various hydrophilic ingredients (e.g., perfumes or hydrophilic care substances) into the agent. For example, said hydrophilic ingredients can be first predissolved in a little water and then emulsified or dispersed in the fatty constituents with the aid of surfactants. In this case, the fatty constituents simultaneously act as a carrier, and a W/O emulsion or an O/W emulsion is formed.

Additional ingredients or active ingredients that contain certain percentages of water can also be used in agent (a). In this context, it has been found that a water content of up to about 10 wt % does not impair the storage stability of agent (a) to a large extent.

Agent (a) can additionally contain further ingredients or active ingredients. In particular, the use of non-ionic surfactants in agent (a) has proven especially advantageous. For example, the one or more non-ionic surfactants can be used in total amounts of from about 0.1 to about 15.0 wt %, from about 0.5 to about 12.5 wt %, from about 1.0 to about 10.0 wt %, or from about 1.5 to about 8.0 wt %, with respect to the total weight of agent (a).

Therefore, also preferred is a multi-component packaging unit (kit of parts) for reductively decoloring dyed keratin fibers that is provided wherein

agent (a) in container (A) additionally contains one or more non-ionic surfactants in a total amount of from about 0.1 to about 15.0 wt %, from about 0.5 to about 12.5 wt %, from about 1.0 to about 10.0 wt %, or from about 1.5 to about 8.0 wt %, with respect to the total weight of agent (a).

The term “surfactants” is understood to mean amphiphilic (bifunctional) compounds having at least one hydrophobic residue and at least one hydrophilic molecule part. The hydrophobic molecule part is usually a hydrocarbon chain having 10 to 30 carbon atoms. In the case of non-ionic surfactants, the hydrophilic molecule part comprises an uncharged, strongly polar structural unit.

Non-ionic surfactants contain, for example, a polyol group, a polyalkylene glycol ether group, or a combination of a polyol group and a polyglycol ether group as a hydrophilic group. Such compounds are, for example,

products of the addition of 2 to 50 mol of ethylene oxide and/or 2 to 50 mol of propylene oxide to linear and branched fatty alcohols having 12 to 30 C atoms, the fatty alcohol polyglycol ethers or the fatty alcohol polypropylene glycol ethers or mixed fatty alcohol polyethers,

products of the addition of 2 to 50 mol of ethylene oxide and/or 2 to 50 mol of propylene oxide to linear and branched fatty acids having 6 to 30 C atoms, the fatty acid polyglycol ethers or the fatty acid polypropylene glycol ethers or mixed fatty acid polyethers,

products of the addition of 2 to 50 mol of ethylene oxide and/or 2 to 50 mol of propylene oxide to linear and branched alkyl phenols having 8 to 15 C atoms in the alkyl group, the alkyl phenol polyglycol ethers or the alkyl polypropylene glycol ethers or mixed alkyl phenol polyethers,

products of the addition of 2 to 50 mol of ethylene oxide and/or 2 to 50 mol of propylene oxide to linear and branched fatty alcohols having 8 to 30 C atoms, to fatty acids having 8 to 30 C atoms, and to alkyl phenols having 8 to 15 C atoms in the alkyl group, said products being end-capped with a methyl residue or C₂-C₆ alkyl residue, such as the types available under the sales names Dehydol® LS and Dehydol® LT (Cognis),

C₁₂-C₃₀ fatty acid mono- and diesters of products of the addition of 2 to 30 mol of ethylene oxide to glycerol,

products of the addition of 5 to 60 mol of ethylene oxide to castor oil and hardened castor oil,

polyol fatty acid esters, such as the commercial product Hydagen® HSP (Cognis) or Sovermol® types (Cognis),

polyalkoxylated triglycerides,

polyalkoxylated fatty acid alkyl esters of formula (Tnio-1),

R¹CO—(OCH₂CHR²)_{w}OR³ (Tnio-1),
in which R^{I}CO represents a linear or branched, saturated and/or unsaturated acyl residue having 6 to 22 carbon atoms, R² represents hydrogen or methyl, R³ represents linear or branched alkyl residues having 1 to 4 carbon atoms, and w represents numbers from 2 to 20,

amine oxides,

hydroxy mixed ethers, which are described, for example, in laid-open application DE 19738866,

sorbitan fatty acid esters and products of the addition of ethylene oxide to sorbitan fatty acid esters, such as the polysorbates,

sugar fatty acid esters and products of the addition of ethylene oxide to sugar fatty acid esters,

products of the addition of ethylene oxide to fatty acid alkanolamides and fatty amines,

sugar surfactants of the type of the alkyl and alkenyl oligoglycosides, or

sugar surfactants of the type of the fatty acid N-alkyl polyhydroxyalkyl amides.

C₁₂-C₃₀ fatty alcohols, C₁₂-C₃₀ fatty acid triglycerides, C₁₂-C₃₀ fatty acid monoglycerides, C₁₂-C₃₀ fatty acid diglycerides, and C₁₂-C₃₀ fatty acid esters do not have a strongly polar terminal group (which is also clear from the low HLBs of the compounds of this group). They are considered fatty constituents in the sense of this invention and therefore are not non-ionic surfactants according to the definition of the present disclosure.

Furthermore, the agents (a) can additionally contain one or more non-ionic polymers. For example, the one or more non-ionic polymers can be used in total amounts of from about 0.1 to about 15.0 wt %, from about 0.2 to about 10.5 wt %, from about 0.25 to about 7.5 wt %, or from about 0.3 to about 5.0 wt %, with respect to the total weight of agent (a).

In a further embodiment, a multi-component packaging unit (kit of parts) as contemplated herein is provided wherein agent (a) in container (A) additionally contains one or more non-ionic polymers in a total amount of from about 0.1 to about 15.0 wt %, from about 0.2 to about 10.5 wt %, from about 0.25 to about 7.5 wt %, or from about 0.3 to about 5.0 wt %, with respect to the total weight of agent (a).

The term “polymers” is understood to mean macromolecules that have a molecular weight of at least about1000 g/mol, preferably at least about 2500 g/mol, especially preferably at least about 5000 g/mol, and that consist of identical, repeating organic units. Polymers are produced by polymerizing one monomer type or by polymerizing different monomer types that differ from each other structurally. If the polymer is produced by polymerizing one monomer type, the term “homopolymer” is used. If structurally different monomer types are used in the polymerization, a person skilled in the art uses the term “copolymer”.

The maximum molecular weight of the polymer depends on the degree of polymerization (number of polymerized monomers) and is determined in part by the polymerization method. According to the present disclosure, it is preferred if the maximum molecular weight of the zwitterionic polymer (d) is not more than 107 g/mol, preferably not more than 106 g/mol, and especially preferably not more than 105 g/mol.

Non-ionic polymers are provided wherein said polymers do not have any charges. This means that non-ionic polymers in the sense of the present disclosure are produced by the homo- or copolymerization of uncharged monomers.

Examples of suitable non-ionic polymers are vinylpyrrolidinone/vinyl acrylate copolymers, polyvinylpyrrolidinone, vinylpyrrolidinone/vinyl acetate copolymers, polyethylene glycols, ethylene/propylene/styrene copolymers, and/or butylene/ethylene/styrene copolymers.

Agent (b) in Container (B)

The multi-component packaging unit as contemplated herein comprises a second container (B), which contains an agent (b). Said agent (b) is a cosmetic agent that contains one or more metal salts from the group of the tin(II) salts, the iron(II) salts, the manganese(II) salts, the titanium(II) salts, and the titanium(III) salts.

As contemplated herein, agent (b) is applied to the keratin fibers as a post-treatment agent after agent (a) has been used. Characteristic of and essential to agent (b) is the content of one or more metal salts from the aforementioned group in agent (b). The metal salts of said group are distinguished in that said metal salts can be converted to a higher oxidation state and in this way have a reducing effect on other compounds. For example, the tin(II) salts can be converted to tin(IV) salts. Iron(II) salts can be converted to iron(III) salts. Manganese(II) salts can be converted to manganese(IV) salts. Titanium(II) and titanium(III) salts can be converted to titanium(IV) salts. The conversion to a higher oxidation state occurs in a redox reaction, in which the other participating reaction partner is reduced. It has been found that the use of agent (b) effectively prevents post-darkening of keratin fibers when agent (b) is used as a post-treatment agent after agent (a) as part of a reductive color removal treatment. In this context, it is assumed that the metal salts, because of the reductive potential thereof, can prevent the reoxidation of the reduced dyes caused by atmospheric oxygen very well.

As contemplated herein, the term “metal salt” is understood to mean a salt of the aforementioned elements, i.e., the metal exists as a cation and is neutralized by the corresponding equivalent of anions, which equivalent is defined by the oxidation state of the metal. The one or more anions can be inorganic or organic. Examples of inorganic anions are chloride, bromite, sulfate, hydrogen sulfate, carbonate, hydrogen carbonate, hydroxide, phosphate, and hydrogen phosphate. Examples of organic anions are formate, acetate, propionate, lactate, citrate, and tartrate.

Within the group of the metal salts as contemplated herein, very specific metal salts have proven especially effective, because said metal salts improve the color removal performance overall and inhibit the post-darkening of the decolored keratin fibers especially well and for an especially long time. Tin(II) salts and iron(II) salts have been found to be especially effective. The tin(II) salts are exceptionally well suited.

In a further, especially preferred embodiment, a multi-component packaging unit (kit of parts) as contemplated herein is therefore provided wherein agent (b) in container (B) contains one or more metal salts from the group of tin(II) chloride, tin(II) bromide, tin(II) fluoride, tin(II) sulfate, tin(II) oxide, tin(II) hydroxide, tin(II) carbonate, tin(II) phosphate, tin(II) acetate, tin(II) gluconate, tin(II) lactate, tin(II) tartrate, tin(II) oxalate, iron(II) chloride, iron(II) bromide, iron(II) fluoride, iron(II) sulfate, iron(II) oxide, iron(II) hydroxide, iron(II) carbonate, iron(II) phosphate, iron(II) acetate, iron(II) gluconate, iron(II) lactate, iron(II) tartrate, and iron(II) oxalate.

Tin(II) chloride has the empirical formula SnCl₂.

Tin(II) bromide has the empirical formula SnBr₂.

Tin(II) fluoride has the empirical formula SnF₂.

Tin(II) sulfate has the empirical formula SnSO4.

Tin(II) oxide has the empirical formula SnO.

Tin(II) hydroxide has the empirical formula Sn(OH)2.

Tin(II) carbonate has the empirical formula Sn(CO3).

Tin(II) phosphate is alternatively also called tin(II) pyrophosphate and has the empirical formula Sn2(P2O7).

Tin(II) acetate has the empirical formula Sn(CH3COO2)2.

Tin(II) gluconate is the tin salt of gluconic acid and has the empirical formula Sn(C6H11O7)2.

Tin(II) lactate is the tin salt of lactic acid and has the empirical formula Sn(C3H5O3)2.

Tin(II) tartrate is the tin salt of tartaric acid and has the empirical formula Sn(C4H4O6).

Tin(II) oxalate is the tin salt of oxalic acid and has the empirical formula Sn(C2O4).

Iron(II) chloride has the empirical formula FeCl2.

Iron(II) bromide has the empirical formula FeBr2.

Iron(II) fluoride has the empirical formula FeF2.

Iron(II) sulfate has the empirical formula Fe(SO4).

Iron(II) oxide has the empirical formula FeO.

Iron(II) hydroxide has the empirical formula Fe(OH)2.

Iron(II) carbonate has the empirical formula Fe(CO3).

Iron(II) phosphate has the empirical formula Fe3(PO4)2.

Iron(II) acetate has the empirical formula Fe(CH3COO2)2.

Iron(II) gluconate is the iron salt of gluconic acid and has the empirical formula Fe(C6H11O7)2.

Iron(II) lactate is the iron salt of lactic acid and has the empirical formula Fe(C3H5O3)2.

Iron(II) tartrate is the iron salt of tartaric acid and has the empirical formula Fe(C4H4O6).

Iron(II) oxalate is the iron salt of oxalic acid and has the empirical formula Fe(C2O4).

The hydrates of the aforementioned metal salts are also contemplated herein.

It was possible to achieve the best effects with an agent (b) that contains one or more tin(II) salts. It was possible to observe the best decoloring performance if an agent (b) that contained one or more salts from the group of tin(II) chloride, tin(II) bromide, tin(II) fluoride, tin(II) acetate, tin(II) gluconate, tin(II) lactate, tin(II) tartrate, and tin(II) oxalate was applied to the keratin fibers after the use of agent (a). The hair that was decolored with a sulfur-containing reductant (a) and then post-treated with tin(II) salts (b) was decolored most intensely in comparison with the dyed hair. If the strands decolored in such a way were stored in air, very little recoloring or reoxidation occurred.

An exceedingly preferred multi-component packaging unit (kit of parts) is therefore provided wherein agent (b) in container (B) contains one or more metal salts from the group of tin(II) chloride, tin(II) bromide, tin(II) fluoride, tin(II) acetate, tin(II) gluconate, tin(II) lactate, tin(II) tartrate, and tin(II) oxalate.

The one or more metal salts are used in agent (b) as contemplated herein preferably in certain amount ranges. Agent (b) preferably contains one or more metal salts in a total amount of from about 0.5 to about 15.0 wt %, from about 1.0 to about 13.0 wt %, from about 2.0 to about 11.0 wt %, or from about 3.0 to about 10.0 wt %. The amount specifications here refer to the total weight of all metal salts as contemplated herein that are contained in agent (b), which total weight is set in relation to the total weight of agent (b).

An exceedingly preferred multi-component packaging unit (kit of parts) is therefore provided wherein agent (b) in container (B) contains the one or more metal salts in a total amount of from about 0.5 to about 15.0 wt %, from about 1.0 to about 13.0 wt %, from about 2.0 to about 11.0 wt %, or from about 3.0 to about 10.0 wt %, with respect to the total weight of agent (b).

An exceedingly preferred multi-component packaging unit (kit of parts) is provided wherein agent (b) in container (B) contains from about 0.5 to about 15.0 wt % of tin(II) chloride, with respect to the total weight of agent (b).

An exceedingly preferred multi-component packaging unit (kit of parts) is therefore provided wherein agent (b) in container (B) contains from about 1.0 to about 13.0 wt % of tin(II) chloride, with respect to the total weight of agent (b).

An exceedingly preferred multi-component packaging unit (kit of parts) is therefore provided wherein agent (b) in container (B) contains from about 2.0 to about 11.0 wt % of tin(II) chloride, with respect to the total weight of agent (b).

An exceedingly preferred multi-component packaging unit (kit of parts) is therefore provided wherein agent (b) in container (B) contains from about 3.0 to about 10.0 wt % of tin(II) chloride, with respect to the total weight of agent (b).

An exceedingly preferred multi-component packaging unit (kit of parts) is therefore provided wherein agent (b) in container (B) contains from about 0.5 to about 15.0 wt % of tin(II) bromide, with respect to the total weight of agent (b).

An exceedingly preferred multi-component packaging unit (kit of parts) is therefore provided wherein agent (b) in container (B) contains from about 1.0 to about 13.0 wt % of tin(II) bromide, with respect to the total weight of agent (b).

An exceedingly preferred multi-component packaging unit (kit of parts) is therefore provided wherein agent (b) in container (B) contains from about 2.0 to about 11.0 wt % of tin(II) bromide, with respect to the total weight of agent (b).

An exceedingly preferred multi-component packaging unit (kit of parts) is therefore provided wherein agent (b) in container (B) contains from about 3.0 to about 10.0 wt % of tin(II) bromide, with respect to the total weight of agent (b).

An exceedingly preferred multi-component packaging unit (kit of parts) is therefore provided wherein agent (b) in container (B) contains from about 0.5 to about 15.0 wt % of tin(II) fluoride, with respect to the total weight of agent (b).

An exceedingly preferred multi-component packaging unit (kit of parts) is therefore provided wherein agent (b) in container (B) contains from about 1.0 to about 13.0 wt % of tin(II) fluoride, with respect to the total weight of agent (b).

An exceedingly preferred multi-component packaging unit (kit of parts) is therefore provided wherein agent (b) in container (B) contains from about 2.0 to about 11.0 wt % of tin(II) fluoride, with respect to the total weight of agent (b).

An exceedingly preferred multi-component packaging unit (kit of parts) is therefore provided wherein agent (b) in container (B) contains from about 3.0 to about 10.0 wt % of tin(II) fluoride, with respect to the total weight of agent (b).

An exceedingly preferred multi-component packaging unit (kit of parts) is therefore provided wherein agent (b) in container (B) contains from about 0.5 to about 15.0 wt % of tin(II) acetate, with respect to the total weight of agent (b).

An exceedingly preferred multi-component packaging unit (kit of parts) is therefore provided wherein agent (b) in container (B) contains from about 1.0 to about 13.0 wt % of tin(II) acetate, with respect to the total weight of agent (b).

An exceedingly preferred multi-component packaging unit (kit of parts) is therefore provided wherein agent (b) in container (B) contains from about 2.0 to about 11.0 wt % of tin(II) acetate, with respect to the total weight of agent (b).

An exceedingly preferred multi-component packaging unit (kit of parts) is therefore provided wherein agent (b) in container (B) contains from about 3.0 to about 10.0 wt % of tin(II) acetate, with respect to the total weight of agent (b).

An exceedingly preferred multi-component packaging unit (kit of parts) is therefore provided wherein agent (b) in container (B) contains from about 0.5 to about 15.0 wt % of tin(II) gluconate, with respect to the total weight of agent (b).

An exceedingly preferred multi-component packaging unit (kit of parts) is therefore provided wherein agent (b) in container (B) contains from about 1.0 to about 13.0 wt % of tin(II) gluconate, with respect to the total weight of agent (b).

An exceedingly preferred multi-component packaging unit (kit of parts) is therefore provided wherein agent (b) in container (B) contains from about 2.0 to about 11.0 wt % of tin(II) gluconate, with respect to the total weight of agent (b).

An exceedingly preferred multi-component packaging unit (kit of parts) is therefore provided wherein agent (b) in container (B) contains from about 3.0 to about 10.0 wt % of tin(II) gluconate, with respect to the total weight of agent (b).

An exceedingly preferred multi-component packaging unit (kit of parts) is therefore provided wherein agent (b) in container (B) contains from about 0.5 to about 15.0 wt % of tin(II) lactate, with respect to the total weight of agent (b).

An exceedingly preferred multi-component packaging unit (kit of parts) is therefore provided wherein agent (b) in container (B) contains from about 1.0 to about 13.0 wt % of tin(II) lactate, with respect to the total weight of agent (b).

An exceedingly preferred multi-component packaging unit (kit of parts) is therefore provided wherein agent (b) in container (B) contains from about 2.0 to about 11.0 wt % of tin(II) lactate, with respect to the total weight of agent (b).

An exceedingly preferred multi-component packaging unit (kit of parts) is therefore provided wherein agent (b) in container (B) contains from about 3.0 to about 10.0 wt % of tin(II) lactate, with respect to the total weight of agent (b).

An exceedingly preferred multi-component packaging unit (kit of parts) is therefore provided wherein agent (b) in container (B) contains from about 0.5 to about 15.0 wt % of tin(II) tartrate, with respect to the total weight of agent (b).

An exceedingly preferred multi-component packaging unit (kit of parts) is therefore provided wherein agent (b) in container (B) contains from about 1.0 to about 13.0 wt % of tin(II) tartrate, with respect to the total weight of agent (b).

An exceedingly preferred multi-component packaging unit (kit of parts) is therefore provided wherein agent (b) in container (B) contains from about 2.0 to about 11.0 wt % of tin(II) tartrate, with respect to the total weight of agent (b).

An exceedingly preferred multi-component packaging unit (kit of parts) is therefore provided wherein agent (b) in container (B) contains from about 3.0 to about 10.0 wt % of tin(II) tartrate, with respect to the total weight of agent (b).

An exceedingly preferred multi-component packaging unit (kit of parts) is therefore provided wherein agent (b) in container (B) contains from about 0.5 to about 15.0 wt % of tin(II) oxalate, with respect to the total weight of agent (b).

An exceedingly preferred multi-component packaging unit (kit of parts) is therefore provided wherein agent (b) in container (B) contains from about 1.0 to about 13.0 wt % of tin(II) oxalate, with respect to the total weight of agent (b).

An exceedingly preferred multi-component packaging unit (kit of parts) is therefore provided wherein agent (b) in container (B) contains from about 2.0 to about 11.0 wt % of tin(II) oxalate, with respect to the total weight of agent (b).

An exceedingly preferred multi-component packaging unit (kit of parts) is therefore provided wherein agent (b) in container (B) contains from about 3.0 to about 10.0 wt % of tin(II) oxalate, with respect to the total weight of agent (b).

Agent (b) contains the one or more metal salts preferably in an aqueous cosmetic carrier.

The water content in agent (b) preferably is from about 40.0 to about 99.0 wt %, from about 50.0 to about 97.0 wt %, from about 60.0 to about 95.0 wt %, or from about 70 to about 93.0 wt %. Here, the water content refers to the amount of water present in agent (b), which amount is set in relation to the total weight of agent (b).

In a further, especially preferred embodiment, a multi-component packaging unit (kit of parts) as contemplated herein is provided wherein agent (b) in container (B) has a water content of from about 40.0 to about 99.0 wt %, from about 50.0 to about 97.0 wt %, from about 60.0 to about 95.0 wt %, or from about 70 to about 93.0 wt %, with respect to the total weight of agent (b).

In the course of the work leading to the present disclosure, it was found that the pH value of the post-treatment agent (b) is preferably set so as to be acidic in order to achieve an optimal decoloring effect. The post-treatment agents exhibited the greatest effect at a pH value of less than or equal to 7, preferably less than or equal to 5.8, more preferably less than or equal to 4.8, even more preferably less than or equal to 3.8, and exceedingly preferably less than or equal to 2.8.

In a further, especially preferred embodiment, a multi-component packaging unit (kit of parts) as contemplated herein is provided wherein agent (b) in container (B) has a pH value of from about 1.0 to about 5.8, from about 1.2 to about 4.8, from about 1.3 to about 3.8, or from about 1.4 to about 2.8.

The pH values of the present disclosure were measured with a glass electrode of type N 61 from Schott at a temperature of 22° C.

In principle, the acidic pH value can be set by using various acids. Suitable acids are, for example, citric acid, tartaric acid, malic acid, lactic acid, acetic acid, sulfuric acid, hydrochloric acid, phosphoric acid, methanesulfonic acid, benzoic acid, malonic acid, oxalic acid, and/or 1-hydroxyethane-1,1-diphosphonic acid. The acids from the group of citric acid, tartaric acid, malic acid, lactic acid, methanesulfonic acid, oxalic acid, malonic acid, benzoic acid, hydrochloric acid, sulfuric acid, phosphoric acid, and/or 1-hydroxyethane-1,1-diphosphonic acid are preferred.

Strong inorganic acids, such as hydrochloric acid, sulfuric acid, and/or phosphoric acid, have proven very well suited for setting the strong acidic pH value ranges.

Agent (c) in Container (C)

As contemplated herein, as described previously, the method of reductively decoloring dyed keratin fibers begins with the use of agent (a). With the one or more sulfur-containing reductants, agent (a) in container (A) contains the reactive substances that are responsible for the reductive decomposition of the chromophoric systems of the dyes. Agent (b) is a post-treatment agent, i.e., agents (a) and (b) are not mixed as contemplated herein.

The reductants in agent (a) are reactive compounds which, to a certain extent, can be decomposed or enter into side reactions in the aqueous environment under certain circumstances. To increase the storage stability of agent (a), agent (a) is formulated preferably water-free or with little water, as previously described.

Each of the various sulfur-containing reductants in agent (a) passes through the effect optimum thereof in a certain pH value range. For example, ready-to-use decoloring agents having dithionite salts are preferably set to an acidic pH value, whereas ready-to-use decoloring agents having formamidine sulfinic acid develop the best effect thereof in the alkaline range.

To create the environment in which the sulfur-containing reductants pass through the effect optimum thereof, it can therefore also be preferred that a ready-to-use decoloring agent is first produced from agent (a) before use. For this purpose, the multi-component packaging unit as contemplated herein can also contain a further container (C) having a third cosmetic agent (c). The third cosmetic agent (c) then contains, in an aqueous carrier, the acids or bases best suited for setting the particular optimal pH value. To produce the ready-to-use decoloring agent, agents (a) and (c) are then mixed, and this application mixture is applied to the dyed keratin fibers.

Agent (c) is preferably an aqueous cosmetic agent that can contain one or more acids or one or more bases (alkalizing agents).

In a further, exceedingly preferred embodiment, a multi-component packaging unit (kit of parts) as contemplated herein is therefore provided wherein agent (c) in container (C) additionally contains one or more acids or one or more alkalizing agents.

If, for example, a dithionite salt is selected as a sulfur-containing reductant in agent (a), carrier agent (c) preferably contains an acid. Accordingly, the pH value of the aqueous carrier agent (c) is preferably also set to a value in the acidic range.

Within this embodiment, an especially preferred multi-component packaging unit (kit of parts) is provided wherein
agent (a) in container (A) contains sodium dithionite, zinc dithionite, and/or potassium dithionite, and
agent (c) in container (C) contains
   (c1) water and
   (c2) one or more acids from the group of the inorganic and/or organic acids.

In particular, the acids citric acid, tartaric acid, malic acid, lactic acid, acetic acid, hydroxyethane-1,1-diphosphonic acid, methanesulfonic acid, benzoic acid, oxaloacetic acid, hydrochloric acid, sulfuric acid, phosphoric acid, malonic acid, and/or oxalic acid have been found to be suitable as acids in this context. The quick and complete dissolution of the thionite salts can be ensured by using one or more acids from this group in agent (c).

Within this embodiment, an especially preferred multi-component packaging unit (kit of parts) is provided wherein agent (c) in container (C) contains
(c2) one or more acids from the group consisting of citric acid, tartaric acid, malic acid, lactic acid, acetic acid, hydroxyethane-1,1-diphosphonic acid, methanesulfonic acid, benzoic acid, oxaloacetic acid, hydrochloric acid, sulfuric acid, phosphoric acid, malonic acid, and/or oxalic acid.

Therefore, the pH value of the aqueous agent preferably lies in the acidic range in this embodiment. Preferably, agent (c) is set to a pH value in the range of from about 1.0 to about 6.0, from about 1.2 to about 5.0, from about 1.3 to about 4.5, or from about 1.4 to about 4.0.

Within this embodiment, an exceedingly preferred multi-component packaging unit (kit of parts) is provided wherein
agent (a) in container (A) contains sodium dithionite, zinc dithionite, and/or potassium dithionite, and
agent (c) in container (C) contains
   (c1) water and
   (c2) one or more acids from the group consisting of citric acid, tartaric acid, malic acid, lactic acid, acetic acid, hydroxyethane-1,1-diphosphonic acid, methanesulfonic acid, benzoic acid, oxaloacetic acid, hydrochloric acid, sulfuric acid, phosphoric acid, malonic acid, and/or oxalic acid.

If formamidine sulfinic acid is used as a sulfur-containing reductant in agent (a), it was likewise possible to achieve excellent decoloring results. Formamidine sulfinic acid passes through the effect optimum thereof at pH values in the alkaline range, i.e., the ready-to-use decoloring agent—and thus also agent (c)—preferably contain at least one alkalizing agent.

In this embodiment, an especially preferred multi-component packaging unit (kit of parts) is provided wherein
agent (a) in container (A) contains formamidine sulfinic acid, and
agent (c) in container (C) contains
   (C1′) water and
   (C2′) one or more alkalizing agents.

For example, basic amino acids such as arginine, lysine, ornithine, and/or histidine can be used as alkalizing agents in the cosmetic carrier agent (c).

As contemplated herein, an organic compound that contains at least one amino group that can be protonated and at least one —COOH group or one —SO3H group is considered to be an amino acid. Preferred amino acids are amino carboxylic acids, particularly α-(alpha)-amino carboxylic acids and ω-amino carboxylic acids, wherein a-amino carboxylic acids are especially preferred.

As contemplated herein, the term “alkaline amino acids” is understood to mean amino acids that have an isoelectric point pI of greater than 7.0.

Alkaline a-amino carboxylic acids contain at least one asymmetric carbon atom. In the present disclosure, both possible enantiomers can be used as a specific compound or mixtures thereof can be used, particularly as racemates. However, it is especially advantageous to use the naturally preferably occurring isomeric form, typically in the L-configuration.

The alkaline amino acids are preferably selected from the group consisting of arginine, lysine, ornithine, and histidine, especially preferably from arginine and lysine. In a further, especially preferred embodiment, an agent as contemplated herein is therefore provided wherein the alkalizing agent is an alkaline amino acid from the group of arginine, lysine, ornithine, and/or histidine.

Other suitable alkalizing agents are, for example, sodium hydroxide, potassium hydroxide, sodium carbonate, sodium hydrogencarbonate, potassium carbonate, and potassium hydrogencarbonate.

Furthermore, alkanolamines can also be used as alkalizing agents in agent (c). The alkanolamines that can be used in agent (c) as contemplated herein are preferably selected from primary amines having a C2-C6 alkyl basic structure, which bears at least one hydroxyl group. Preferred alkanolamines are selected from the group consisting of 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, and 2-amino-2-methylpropane-1,3-diol.

Alkanolamines especially preferred as contemplated herein are selected from 2-aminoethan-1-ol and/or 2-amino-2-methylpropan-1-ol. An especially preferred embodiment is therefore provided wherein agent (c) as contemplated herein contains an alkanolamine selected from 2-aminoethan-1-ol and/or 2-amino-2-methylpropan-1-ol as an alkalizing agent.

In this context, nitrogen-containing alkalizing agents from the group consisting of ammonia and/or alkanolamines have been found to be exceedingly suitable. Especially preferred alkanolamines can be selected, for example, from the group consisting of ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, and/or 2-amino-2-methylpropane-1,3-diol.

In this embodiment, an especially preferred multi-component packaging unit (kit of parts) is provided wherein agent (c) in container (C) contains
(c2′) one or more alkalizing agents from the group consisting of ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, and 2-amino-2-methylpropane-1,3-diol.

The pH value of the aqueous agent (c) preferably lies in the alkaline range in this embodiment. Preferably, agent (c) is set to a pH value in the range of from about 7.0 to about 12.0, from about 7.5 to about 11.0, from about 8.0 to about 10.5, or from about 8.0 to about 10.0, in this embodiment.

The ready-to-use decoloring agent can be produced by mixing agents (a) and (c). In principle, agents (a) and (c) can be mixed in various mixture ratios, such as (a)/(c) of 20:1 to 1:20.

Agent (a) is preferably a low-water, solid, powdery, particularly preferably pasty agent. However, it is advantageous to use agent (c) at least in the same amount as agent (a) so that agent (a) also can be completely dissolved when mixed with agent (c). It is also preferred that agent (c) is used in excess.

In a further, preferred embodiment, a multi-component packaging unit as contemplated herein is therefore provided wherein the amounts of agent (a) in container (A) and of agent (c) in container (C) are selected in such a way that, when the application mixture is produced, i.e., when agents (a) and (c) are mixed, the mixture ratio (a)/(c) is from about 1:5 to about 5:1, from about 1:3 to about 3:1, or from about 1:2 to about 2:1.

For example, agent (a) can be completely transferred from container (A) into container (C), which already contains agent (c), to produce the mixture. In this case, the size of container (C) is selected in such a way that container (C) can hold the total amount of agents (a) and (c) and also permits mixing of the two agents (a) and (c), for example by shaking or stirring.

Analogously, the mixture can be produced also by completely transferring agent (c) from container (C) into container (A), which already contains agent (a). In this case, the size of container (A) should be selected in such a way that container (A) can hold the total amount of agents (a) and (c) and also permits mixing of the two agents (a) and (c), for example by shaking or stirring.

A further possibility for producing the application mixture is to completely transfer both agents (a) and (c) from containers (A) and (C) into a third vessel, which then permits the mixing of the two agents, for example by shaking or stirring.

Decoloring of Dyed Keratin Fibers

The multi-component packaging unit as contemplated herein is a product comprising agents (a) and (b) (and optionally further components such as agent (c)) that is used to decolor previously dyed keratin fibers, particularly human hair. The dyed keratin fibers are usually fibers that were previously dyed with conventional oxidation dyes and/or substantive dyes known to a person skilled in the art.

The decoloring agents are suitable for removing colorings that were produced on the keratin fibers by employing oxidation dyes based on developer and coupler components. If the following compounds were used as developers, the colorings produced by means thereof can be removed well, effectively, and nearly without later post-darkening by using the decoloring agent: p-phenylenediamine, p-toluylenediamine, N,N-bis(β-hydroxyethyl)-p-phenylenediamine, 4-N,N-bis(β-hydroxyethyl)-amino-2-methylaniline, 2-(β-hydroxyethyl)-p-phenylenediamine, 2-(α,β-dihydroxyethyl)-p-phenylenediamine, 2-hydroxymethyl-p-phenylenediamine, bis(2-hydroxy-5-aminophenyl)-methane, p-aminophenol, 4-amino-3-methylphenol, 2,4,5,6-tetraaminopyrimidine, 4-hydroxy-2,5,6-triaminopyrimidine, 2-hydroxy-4,5,6-triaminopyrimidine, and/or 4,5-diamino-1-(β-hydroxyethyl)pyrazole.

If the following compounds were used as couplers, the colorings produced by means thereof likewise can be removed with very good decoloring results: m-phenylenediamine derivatives, naphthols, resorcinol and resorcinol derivatives, pyrazolones, and m-aminophenol derivatives. In particular, 1-naphthol, 1,5-, 2,7-, and 1,7-dihydroxynaphthalene, 5-amino-2-methylphenol, m-aminophenol, resorcinol, resorcinol monomethyl ether, m-phenylenediamine, 1-phenyl-3-methylpyrazolone-5, 2,4-dichloro-3-aminophenol, 1,3-bis(2′,4′-diaminophenoxy)propane, 2-chlororesorcinol, 4-chlororesorcinol, 2-chloro-6-methyl-3-aminophenol, 2-amino-3-hydroxypyridine, 2-methylresorcinol, 5-methylresorcinol, and 2-methyl-4-chloro-5-aminophenol are suitable as coupler substances. 1-Naphthol, 1,5-dihydroxynaphthalene, 2,7-dihydroxynaphthalene, 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol, and 2,6-dihydroxy-3,4-dimethylpyridine.

The substrate to be decolored also can have been dyed with substantive dyes. In particular, nitrophenylenediamines, nitroaminophenols, azo dyes, anthraquinones, or indophenols are possible as substantive dyes. Preferred substantive dyes are the compounds known under the internal designations or trade names HC Yellow 2, HC Yellow 4, HC Yellow 5, HC Yellow 6, HC Yellow 12, Acid Yellow 1, Acid Yellow 10, Acid Yellow 23, Acid Yellow 36, HC Orange 1, Disperse Orange 3, Acid Orange 7, HC Red 1, HC Red 3, HC Red 10, HC Red 11, HC Red 13, Acid Red 33, Acid Red 52, HC Red BN, Pigment Red 57:1, HC Blue 2, HC Blue 12, Disperse Blue 3, Acid Blue 7, Acid Green 50, HC Violet 1, Disperse Violet 1, Disperse Violet 4, Acid Violet 43, Disperse Black 9, Acid Black 1, and Acid Black 52, and 1,4-diamino-2-nitrobenzene, 2-amino-4-nitrophenol, 1,4-bis(β-hydroxyethyl)amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-1-hydroxy-4-nitrobenzene.

Furthermore, the substrates to be decolored can also be dyed by employing natural dyes occurring in nature, which are contained, for example, in red henna, neutral henna, black henna, chamomile flower, sandalwood, black tea, alder buckthorn bark, sage, logwood, madder root, catechu, Christ's thorn jujube, and Alkanna root.

The decoloring agents as contemplated herein are intended to remove these colorings and therefore preferably contain no dyes themselves, i.e., no oxidation dye intermediates of the developer type and of the coupler type and also no substantive dyes.

In a further, preferred embodiment, a multi-component packaging unit (kit of parts) as contemplated herein is therefore provided wherein

the total amount of all dyes and oxidation dye intermediates contained in agent (a) is at most 0.2 wt %, preferably at most 0.1 wt %, more preferably at most 0.05 wt %, and especially preferably at most 0.01 wt %, with respect to the total weight of agent (a), and

the total amount of all dyes and oxidation dye intermediates contained in agent (b) is at most 0.2 wt %, preferably at most 0.1 wt %, more preferably at most 0.05 wt %, and especially preferably at most 0.01 wt %, with respect to the total weight of agent (b).

The multi-component packaging unit as contemplated herein is used to reductively decolor dyed keratin fibers. Agents (a) and (b) are used on the keratin fibers in succession. Both agents contain reductive substances. For reasons of incompatibility and to avoid exothermic, uncontrollable reactions, agents (a) and (b) therefore preferably do not contain an oxidant.

Here, the term “oxidants” is understood to mean, in particular, the oxidants that can also be used for oxidative decoloring, such as hydrogen peroxide and persulfates (potassium persulfate (alternatively potassium peroxydisulfate), sodium persulfate (sodium peroxydisulfate), and ammonium persulfate (alternatively ammonium peroxydisulfate)). Therefore, preferably neither of agents (a) and (b) contains the aforementioned oxidants.

In a further, preferred embodiment, a multi-component packaging unit (kit of parts) as contemplated herein is therefore provided wherein

the total amount of all oxidants from the group of the peroxides and persulfates contained in agent (a) is at most 0.2 wt %, preferably at most 0.1 wt %, more preferably at most 0.05 wt %, and especially preferably at most 0.01 wt %, with respect to the total weight of agent (a), and

the total amount of all oxidants from the group of the peroxides and persulfates contained in agent (b) is at most 0.2 wt %, preferably at most 0.1 wt %, more preferably at most 0.05 wt %, and especially preferably at most 0.01 wt %, with respect to the total weight of agent (b).

Furthermore, agents (a) and (b) as contemplated herein (and also the optionally additionally present agent (c)) can contain further active ingredients, auxiliaries, and additives, such as non-ionic polymers such as vinylpyrrolidinone/vinyl acrylate copolymers, polyvinylpyrrolidinone, vinylpyrrolidinone/vinyl acetate copolymers, polyethylene glycols, and polysiloxanes; additional silicones such as volatile or non-volatile, straight-chain, branched, or cyclic, crosslinked or uncrosslinked polyalkylsiloxanes (such as dimethicone or cyclomethicone), polyarylsiloxanes, and/or polyalkylarylsiloxanes, particularly polysiloxanes having organofunctional groups, such as substituted or unsubstituted amines (amodimethicone), carboxyl, alkoxy, and/or hydroxyl groups (dimethicone copolyols), linear polysiloxane (A)/polyoxyalkylene (B) block copolymers, graft silicone polymers; cationic polymers such as quaternized cellulose ethers, polysiloxanes having quaternary groups, dimethyldiallylammonium chloride polymers, acrylamide/dimethyldiallylammonium chloride copolymers, dimethylamino ethylmethacrylate/vinylpyrrolidinone copolymers quaternized with diethyl sulfate, vinylpyrrolidinone/imidazolinium methochloride copolymers, and quaternized polyvinyl alcohol; zwitterionic and amphoteric polymers; anionic polymers such as polyacrylic acids or crosslinked polyacrylic acids; structurants such as glucose, maleic acid, and lactic acid; hair-conditioning compounds such as phospholipids, for example lecithin and cephalins; perfume oils, dimethyl isosorbide and cyclodextrins; fiber-structure-improving active ingredients, particularly mono-, di-, and oligosaccharides such as glucose, galactose, fructose, fruit sugar, and lactose; dyes for tinting the agent; anti-dandruff active ingredients such as piroctone olamine, Zinc Omadine, and climbazole; amino acids and oligopeptides; animal- and/or plant-based protein hydrolysates, including in the form of the fatty acid condensation products thereof or optionally anionically or cationically modified derivatives thereof; plant oils; light stabilizers and UV blockers; active ingredients such as panthenol, pantothenic acid, pantolactone, allantoin, pyrrolidinone carboxylic acids and salts thereof, and bisabolol; polyphenols, particularly hydroxycinnamic acids, 6,7-dihydroxycumarins, hydroxybenzoic acids, catechins, tannins, leucoanthocyanidins, anthocyanidins, flavanones, flavones, and flavonols; ceramides or pseudo-ceramides; vitamins, provitamins, and vitamin precursors; plant extracts; fats and waxes such as fatty alcohols, beeswax, montan wax, and paraffins; swelling and penetrating substances such as glycerol, propylene glycol monoethyl ether, carbonates, hydrogencarbonates, guanidines, ureas, and primary, secondary, and tertiary phosphates; opacifying agents such as latex, styrene/PVP copolymers, and styrene/acrylamide copolymers; pearlescing agents such as ethylene glycol mono- and distearate and PEG-3 distearate; pigments; and propellants such as propane-butane mixtures, N₂O, dimethyl ether, CO₂, and air. In this context, the known monographs, e.g., Kh. Schrader, “Grundlagen and Rezepturen der Kosmetika”, 2nd edition, Hüthig Buch Verlag, Heidelberg, 1989, that represent the relevant knowledge of a person skilled in the art are expressly referenced.

The multi-component packaging unit as contemplated herein can additionally contain one or more further agents in further separately produced containers. For example, the multi-component packaging unit as contemplated herein can also contain a container (D) containing a cosmetic agent (d). Said agent (d) can be, for example, a pretreatment agent or a care agent.

Method

As contemplated herein, the previously described multi-component packaging units (kits of parts) are used in methods for reductively decoloring dyed keratin fibers.

A second subject of the present disclosure is a method for reductively decoloring dyed keratin fibers, comprising the following steps in the indicated order:
(I) producing a ready-to-use decoloring agent by mixing an agent (a), as it was disclosed in detail in the description of the first subject of the invention, with an agent (c), as it was disclosed in detail in the description of the first subject of the invention,
(II) applying the ready-to-use decoloring agent to keratin fibers,
(III) allowing the decoloring agent to act, preferably for a time span of from about 5 to about 120 minutes, from about 10 to about 100 minutes, from about 20 to about 80 minutes, or from about 30 to about 65 minutes,
(IV) optionally rinsing the decoloring agent off of the keratin fibers,
(V) applying a cosmetic agent (b) to the keratin fibers, wherein agent (b) is an agent as was disclosed in detail in the description of the first subject of the invention,
(VI) allowing agent (b) to act, preferably for a time span of from about 30 seconds to about 120 minutes, from about 30 seconds to about 45 minutes, from from 30 seconds to from 20 minutes, or from about 30 seconds to about 10 minutes, and
(VII) optionally rinsing agent (b) off of the keratin fibers.

In other words, a second subject of the present disclosure is a method for reductively decoloring dyed keratin fibers, comprising the following steps in the indicated order:
(I) producing a ready-to-use decoloring agent by mixing an agent (a) with an agent (c),
(II) applying the ready-to-use decoloring agent to keratin fibers,
(III) allowing the decoloring agent to act, preferably for a time span of from about 5 to about 120 minutes, from about 10 to about 100 minutes, from about 20 to about 80 minutes, or from about 30 to about 65 minutes,
(IV) optionally rinsing the decoloring agent off of the keratin fibers,
(V) applying a cosmetic agent (b) to the keratin fibers,
(VI) allowing agent (b) to act, preferably for a time span of from about 30 seconds to about 120 minutes, from about 30 seconds to about 45 minutes, from about 30 seconds to about 20 minutes, or from about 30 seconds to about 10 minutes, and
(VII) optionally rinsing agent (b) off of the keratin fibers, wherein

agent (a) contains one or more sulfur-containing reductants, and

agent (b) contains one or more metal salts from the group of the tin(II) salts, the iron(II) salts, the manganese(II) salts, the titanium(II) salts, and the titanium(III) salts, and

agent (c) has a water content of from about 40.0 to about 99.0 wt %, from about 50.0 to about 99.0 wt %, from about 60.0 to about 99.0 wt %, or from about 70 to about 99.0 wt %, with respect to the total weight of agent (c).

Steps (I), (II), and (III) of the method constitute the decoloring process of the keratin fibers and are therefore performed one immediately after the other.

If the decoloring agent should be rinsed off in step (IV), step (IV) occurs immediately after step (III).

In principle, there is no time limitation for the sequence of steps (V), (VI), and (VII). Thus, step (V) can occur hours, days, or, for example, even up to two weeks after the conclusion of step (IV) (or after the conclusion of step (III) if rinsing out should not occur).

It is also possible and as contemplated herein if the decoloring steps (I) to (VII) are performed multiple times one after the other.

As previously described, agents (a) and (c) are preferably used in an amount ratio (a)/(c) that is from about 1:5 to about 5:1, from about 1:3 to about 3:1, or from about 1:2 to about 2:1.

Therefore, also preferred is a method for decoloring dyed keratin fibers that is provided wherein the production of the ready-to-use decoloring agent in step (I) is performed by mixing agent (a) with agent (c), wherein the two agents are used in an amount ratio (a)/(c) having a value of from about 1:5 to about 5:1, from about 1:3 to 3:1, or from about 1:2 to about 2:1.

A post-treatment agent can be used, in particular, to improve the overall decoloring performance and to prevent redarkening or reoxidation, which is caused by the action of atmospheric oxygen on the decolored keratin fibers. To prevent this reoxidation effectively, the post-treatment should occur before the atmospheric oxygen has time to act on the reduced keratin fibers. For this reason, the post-treatment should occur immediately after the decoloring (i.e., immediately after the conclusion of step (IV)), if possible. It is therefore especially preferred if a time span of at most 12 hours, preferably at most 6 hours, more preferably at most 1 hour, and especially preferably at most 30 minutes, lies between the conclusion of step (IV) and the start of step (V).

A preferred method as contemplated herein is therefore provided wherein a time span of at most about 12 hours, preferably at most about 6 hours, more preferably at most about 1 hour, and especially preferably at most about 30 minutes, lies between steps (IV) and (V).

Especially preferred is a method for reductively decoloring dyed keratin fibers, comprising the following steps in the indicated order:
(I) producing a ready-to-use decoloring agent by mixing an agent (a), as it was disclosed in detail in the description of the first subject of the invention, with an agent (c), as it was disclosed in detail in the description of the first subject of the invention,
(II) applying the ready-to-use decoloring agent to keratin fibers,
(III) allowing the decoloring agent to act, preferably for a time span of from about 5 to about 120 minutes, from about 10 to about 100 minutes, from about 20 to about 80 minutes, or from about 30 to about 65 minutes,
(IV) rinsing the decoloring agent off of the keratin fibers,
(V) applying a cosmetic agent (b) to the keratin fibers, wherein agent (b) is an agent as was disclosed in detail in the description of the first subject of the invention,
(VI) allowing agent (b) to act, preferably for a time span of from about 30 seconds to about 120 minutes, from about 30 seconds to about 45 minutes, from about 30 seconds to about 20 minutes, or from about 30 seconds to about 10 minutes, and
(VII) rinsing agent (b) off of the keratin fibers.

The use of the post-treatment agent (b) can also be repeated several times, for example if the post-treatment agent (b) is provided in the form of a shampoo that is used regularly after the decoloring. In this embodiment, agent (b) preferably additionally contains one or more surfactants. If the post-treatment, i.e., the performance of steps (V) to (VII), is repeated, it becomes possible to suppress the reoxidation for a particularly long time.

In a further, preferred embodiment, the post-treatment agent (b) can also be formulated as a leave-on product, which remains on the keratin fibers after the use thereof. If agent (b) is designed as a leave-on product, agent (b) preferably contains no or only small amounts of surfactants.

Therefore, also especially preferred is a method for reductively decoloring dyed keratin fibers, comprising the following steps in the indicated order:
(I) producing a ready-to-use decoloring agent by mixing an agent (a), as it was disclosed in detail in the description of the first subject of the invention, with an agent (c), as it was disclosed in detail in the description of the first subject of the invention,
(II) applying the ready-to-use decoloring agent to keratin fibers,
(III) allowing the decoloring agent to act, preferably for a time span of from about 5 to about 120 minutes, from about 10 to about 100 minutes, from about 20 to about 80 minutes, or from about 30 to about 65 minutes,
(IV) rinsing the decoloring agent off of the keratin fibers,
(V) applying a cosmetic agent (b) to the keratin fibers, wherein agent (b) is an agent as was disclosed in detail in the description of the first subject of the invention,
(VI) allowing agent (b) to act, and
(VII) not rinsing agent (b) off of the keratin fibers.

Because agent (b) is not rinsed out in this embodiment, the method as contemplated herein ends with step (VI) in principle. Therefore, preferred is a method for reductively decoloring dyed keratin fibers, comprising the following steps in the indicated order:
(I) producing a ready-to-use decoloring agent by mixing an agent (a), as it was disclosed in detail in the description of the first subject of the invention, with an agent (c), as it was disclosed in detail in the description of the first subject of the invention,
(II) applying the ready-to-use decoloring agent to keratin fibers,
(III) allowing the decoloring agent to act, preferably for a time span of from about 5 to about 120 minutes, from about 10 to about 100 minutes, from about 20 to about 80 minutes, or from about 30 to about 65 minutes,
(IV) rinsing the decoloring agent off of the keratin fibers,
(V) applying a cosmetic agent (b) to the keratin fibers, wherein agent (b) is an agent as was disclosed in detail in the description of the first subject of the invention,
(VI) allowing agent (b) to act.

The statements made with regard to the multi-component packaging unit as contemplated herein apply, mutatis mutandis, to further, preferred embodiments of the method as contemplated herein.

### EXAMPLES

1.1. Coloring

The following formulations were produced (all specifications in wt %):

Dyeing Cream (F1)

| | wt % |
|---|---|
| Cetearyl alcohol | 3.4 |
| C12-C18 fatty alcohols | 1.2 |
| Ceteareth-20 | 0.3 |
| Ceteareth-12 | 0.3 |
| Lamesoft PO 65 (coco-glucoside, glyceryl oleate, water) | 1.00 |
| Sodium laureth-6 carboxylate (21% aqueous solution) | 4.91 |
| Sodium myreth sulfate (68-73% aqueous solution) | 1.38 |
| Paraffinum liquidum | 0.29 |
| Sodium acrylate/trimethylammoniopropylacrylamide | 1.84 |
| chloride copolymer (19-21% aqueous solution) | |
| p-Toluylenediamine sulfate | 0.88 |
| Resorcinol | 0.30 |
| 2-Methylresorcinol | 0.04 |
| 2-Amino-3-hydroxypyridine | 0.05 |
| m-Aminophenol | 0.05 |
| Sodium sulfite | 0.2 |
| Ascorbic acid | 0.05 |
| 1-Hydroxyethane-1,1-diphosphonic acid (60% aqueous solution) | 0.1 |
| Sodium hydroxide (50% aqueous solution) | 0.54 |
| Sodium silicate 40/42 | 0.25 |
| Ammonia (25% aqueous solution) | 3.19 |
| Water | Ad 100 |

### Oxidant (Ox)

| | wt % |
|---|---|
| Sodium hydroxide | 0.39 |
| Dipicolinic acid | 0.05 |
| Disodium pyrophosphate | 0.02 |
| Sodium laureth sulfate (3 EO) | 1.02 |
| 1-Hydroxyethane-1,1-diphosphonic acid (60% aqueous solution) | 0.76 |
| Aculyn 33 A (acrylates copolymer) | 7.63 |
| Hydrogen peroxide (50% aqueous solution) | 6.1 |
| Water | Ad 100 |

The dyeing cream (F1) and the oxidant (Ox) were mixed in an amount ratio of 1:1 and applied to hair strands (Kerling European natural hair, white). The weight ratio of application mixture to hair was 4:1, and the exposure time was 30 minutes at a temperature of 32 degrees Celsius. Then the strands were rinsed with water, dried, and allowed to rest at room temperature for at least 24 hours. The strands were dyed in a medium-brown shade of color.

The hair was colorimetrically measured.

1.2. Decoloring

The following decoloring agents were produced (all specifications in wt % active substance):

Agent (a)

| Agent (a) | wt % |
|---|---|
| Versagel M 1600⁽¹⁾ | 4.25 |
| Lanette N⁽²⁾ | 6.00 |
| Ceteareth-20 (C16-C18 fatty alcohol, ethoxylated with 20 | 0.50 |
| EO) | |
| Ceteareth-50 (C16-C18 fatty alcohol, ethoxylated with 50 | 2.75 |
| EO) | |
| Formamidine sulfinic acid | 50 |
| Paraffinum liquidum | Ad 100 |

⁽¹⁾INCI: Paraffinum Liquidum (Mineral Oil), Ethylene/Propylene/Styrene Copolymer, Butylene/Ethylene/Styrene Copolymer
⁽²⁾INCI: Cetearyl Alcohol (approx. 90%) and Sodium Cetearyl Sulfate (approx. 10.0%)

Agent (c)

| Agent (c) | wt % |
|---|---|
| Monoethanolamine | 2.0 |
| Emulgade F⁽³⁾ | 3.0 |
| Water (distilled) | Ad 100 |

⁽³⁾CETEARYL ALCOHOL, PEG-40 CASTOR OIL, SODIUM CETEARYL SULFATE

Agents (a) and (c) were mixed with each other at an amount ratio (a)/(c) of 1:4 (i.e., 20 g of agent (a) and 80 g of agent (c)).

The ready-to-use decoloring agents ((a)+(c)) produced in this way were each applied to the hair colored according to point 1.1 and allowed to act for 30 minutes. Thereafter, the strands were rinsed with water for 20 seconds.

One of the following post-treatment agents (agent (b)) was then applied to the still moist strands. The post-treatment agent (b) was allowed to act for 30 minutes. Thereafter, the strands were rinsed with water and dried in air for 2 days.

(V=comparison; E=as contemplated herein)

Agent (b) (all specifications in wt %)

| | (b) V | (b) E1 | (b) E2 | (b) E3 | (b) E4 | (b) E5 |
|---|---|---|---|---|---|---|
| Tin(II) fluoride | — | 5.0 | 5.0 | — | — | — |
| (SnF₂) | | | | | | |
| Tin(II) chloride | — | — | — | 5.0 | — | — |
| (SnCl₂) | | | | | | |
| Iron(II) chloride | — | — | — | — | 5.0 | 5.0 |
| (FeCl₂) | | | | | | |
| Sulfuric acid | pH 2.3 | pH 2.3 | pH 1.5 | pH 1.5 | pH 2.8 | pH 1.5 |
| Emulgade F⁽³⁾ | 3.0 | 3.0 | 3.0 | 3.0 | 3.0 | 3.0 |
| Water | Ad | Ad | Ad | Ad | Ad | Ad |
| | 100 | 100 | 100 | 100 | 100 | 100 |

⁽³⁾Emulgade F: CETEARYL ALCOHOL, PEG-40 CASTOR OIL, SODIUM CETEARYL SULFATE

The hair was colorimetrically measured again. To determine the decoloring effect, the ΔL value was determined in accordance with the following formula.

ΔL=L(after the decoloring)−L(after the coloring)

Within the Lab color space, the L axis describes the lightness of a color (L=0 means black, L=100 means white). The greater the ΔL value is, the greater the lightness difference between dyed and decolored strands is and the more intensely the hair is decolored. The higher the ΔL value is, the better effect a decoloring agent has.

| | L | a | b | ΔL |
|---|---|---|---|---|
| (b) V (pH 2.3) | 21.69 | 7.38 | 7.71 | 4.6 |
| (b) E1 (SnF₂, pH 2.3) | 42.61 | 9.51 | 22.49 | 25.5 |
| (b) E2 (SnF₂, pH 1.5) | 42.18 | 10.41 | 22.23 | 25.1 |
| (b) E3 (SnCl₂, pH 1.5) | 42.60 | 9.35 | 21.44 | 25.5 |
| (b) E4 (FeCl₂, pH 2.8) | 22.64 | 5.51 | 8.09 | 5.5 |
| (b) E5 (FeCl₂, pH 1.5) | 25.93 | 8.01 | 11.26 | 8.8 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A multi-component packaging unit for reductively decoloring dyed keratin fibers, which comprises:
a container (A) containing including a cosmetic agent (a) and
a container (B) including containing a cosmetic agent (b),
which are produced separately from each other, wherein
agent (a) in container (A) comprises one or more sulfur-containing reductants and
agent (b) in container (B) comprises one or more metal salts chosen from the group of tin(II) salts, iron(II) salts, manganese(II) salts, titanium(II) salts, or titanium(III) salts.

2. The multi-component packaging unit (kit of parts) according to claim 1, wherein agent (a) in container (A) comprises one or more sulfur-containing reductants chosen from the group of (H₂N)(NH)C(SO₂H) formamidine sulfinic acid, sodium dithionite, zinc dithionite, potassium dithionite, sodium sulfite, sodium hydrogen sulfite, potassium sulfite, potassium hydrogen sulfite, ammonium sulfite, sodium thiosulfate, potassium thiosulfate, ammonium thiosulfate, hydroxymethanesulfinic acid, aminomethanesulfinic acid, cysteine, thiolactic acid, thioglycolic acid, HN(CH₂SO₂Na)₂ disodium [(sulfinatomethyl)amino]methanesulfinate, HN(CH₂SO₂K)₂ dipotassium [(sulfinatomethyl)amino]methanesulfinate, HN(CH₂SO₂H)₂ [(sulfinomethyl)amino]methanesulfinic acid, N(CH₂SO₂Na)₃ trisodium [bis(sulfinatomethyl)amino]methanesulfinate, N(CH₂SO₂K)₃ tripotassium [bis(sulfinatomethyl)amino]methanesulfinate, N(CH₂SO₂H)₃ [bis(sulfinomethyl)amino]methanesulfinic acid, H₂NCH(CH₃)SO₂Na sodium 1-aminoethane-1-sulfinate, H₂NCH(CH₃)SO₂K potassium 1-aminoethane-1-sulfinate, H₂NCH(CH₃)SO₂H 1-aminoethane-1-sulfinic acid, HN(CH(CH₃)SO₂Na)₂ disodium 1-[(1-sulfinatoethyl)amino]ethane-1-sulfinate, HN(CH(CH₃)SO₂K)₂ dipotassium 1-[(1-sulfinatoethyl)amino]ethane-1-sulfinate, HN(CH(CH₃)SO₂H)₂ 1-[(1-sulfinoethyl)amino]ethane-1-sulfinic acid, N(CH(CH₃)SO₂Na)₃ trisodium 1-[bis(1-sulfinatoethyl)amino]ethane-1-sulfinate, N(CH(CH₃)SO₂K)₃ tripotassium 1-[bis(1-sulfinatoethyl)amino]ethane-1-sulfinate, or N(CH(CH₃)SO₂H)₃ 1-[bis(1-sulfinoethyl)amino]ethane-1-sulfinic acid.

3. The multi-component packaging unit according to claim 1, wherein agent (a) in container (A) comprises one or more sulfur-containing reductants chosen from the group of (H₂N)(NH)C(SO₂H) formamidine sulfinic acid, sodium dithionite, zinc dithionite, N(CH₂SO₂Na)₃ trisodium [bis(sulfinatomethyl)amino]methanesulfinate, N(CH₂SO₂K)₃ tripotassium [bis(sulfinatomethyl)amino]methanesulfinate, or N(CH₂SO₂H)₃ [bis(sulfinomethyl)amino]methanesulfinic acid.

4. The multi-component packaging unit according to claim 1, wherein agent (a) in container (A) comprises one or more sulfur-containing reductants in a total amount of from about 0.1 to about 80.0 wt %, with respect to the total weight of agent (a).

5. The multi-component packaging unit according to claim 1, wherein agent (a) in container (A) comprises one or more fatty constituents in a total amount of from about 5.0 to about 45.0 wt %, with respect to the total weight of agent (a).

6. The multi-component packaging unit according to claim 1, wherein agent (a) in container (A) has a water content of at most about 10.0 wt %, with respect to the total weight of agent (a).

7. The multi-component packaging unit according to claim 1, wherein agent (b) in container (B) comprises one or more metal salts chosen from the group of tin(II) chloride, tin(II) bromide, tin(II) fluoride, tin(II) sulfate, tin(II) oxide, tin(II) hydroxide, tin(II) carbonate, tin(II) phosphate, tin(II) acetate, tin(II) gluconate, tin(II) lactate, tin(II) tartrate, tin(II) oxalate, iron(II) chloride, iron(II) bromide, iron(II) fluoride, iron(II) sulfate, iron(II) oxide, iron(II) hydroxide, iron(II) carbonate, iron(II) phosphate, iron(II) acetate, iron(II) gluconate, iron(II) lactate, iron(II) tartrate, and or iron(II) oxalate.

8. The multi-component packaging unit according to claim 1, wherein agent (b) in container (B) comprises one or more metal salts chosen from the group of tin(II) chloride, tin(II) bromide, tin(II) fluoride, tin(II) acetate, tin(II) gluconate, tin(II) lactate, or tin(II) tartrate.

9. The multi-component packaging unit according to claim 1, wherein agent (b) in container (B) comprises the one or more metal salts in a total amount of from about 0.5 to about 15.0 wt %, with respect to the total weight of agent (b).

10. The multi-component packaging unit according to claim 1, wherein agent (b) in container (B) has a water content of from about 40.0 to about 99.0 wt %, with respect to the total weight of agent (b).

11. The multi-component packaging unit according to claim 1, wherein agent (b) in container (B) has a pH value of from about 1.0 to about 5.8.

12. The multi-component packaging unit according to claim 1, wherein said multi-component packaging unit further comprises:
a container (C) including a cosmetic agent (c), which container (C) is produced separately,
wherein agent (c) has a water content of from about 40.0 to about 99.0 wt %, with respect to the total weight of agent (c).

13. The multi-component packaging unit according to claim 1, wherein agent (c) in container (C) additionally comprises one or more acids.

14. A method for reductively decoloring dyed keratin fibers, comprising the following steps in the indicated order:
(I) producing a ready-to-use decoloring agent by mixing an agent (a) comprising one or more sulfur-containing reductants, with a cosmetic agent (c) having a water content of from about 40.0 to about 99.0 wt % and comprising one or more acids or one or more alkalizing agents,
(II) applying the ready-to-use decoloring agent to keratin fibers,
(III) allowing the decoloring agent to act,
(IV) optionally rinsing the decoloring agent off of the keratin fibers,
(V) applying a cosmetic agent (b) to the keratin fibers, wherein agent (b) comprises one or more metal salts chosen from the group of tin(II) salts, iron(II) salts, manganese(II) salts, titanium(II) salts, or titanium(III) salts,
(VI) allowing agent (b) to act, and
(VII) optionally rinsing agent (b) off of the keratin fibers.

15. The multi-component packaging unit according to claim 1, wherein agent (a) in container (A) comprises:
one or more sulfur-containing reductants in a total amount of from about 35.0 to about 55.0 wt %, with respect to the total weight of agent (a),
one or more fatty constituents in a total amount of from about 5.0 to about 15.0 wt %, with respect to the total weight of agent (a),
wherein agent (a) in container (A) has a water content of at most about 1.0 wt %, with respect to the total weight of agent (a).

16. The multi-component packaging unit according to claim 1, wherein agent (b) in container (B) comprises:
the one or more metal salts in a total amount of from about 3.0 to about 10.0 wt %, with respect to the total weight of agent (b),
wherein agent (b) in container (B) has a water content of from about 70 to about 93.0 wt %, with respect to the total weight of agent (b).

17. The multi-component packaging unit according to claim 1, wherein agent (b) in container (B) has a pH value of from about 1.4 to about 2.8.

18. The multi-component packaging unit according to claim 12, wherein agent (c) has a water content of from about 70 to about 99.0 wt %, with respect to the total weight of agent (c).

19. The multi-component packaging unit according to claim 1, wherein agent (c) in container (C) additionally comprises one or more alkalizing agents.

20. A multi-component packaging unit for reductively decoloring dyed keratin fibers, which comprises:
a container (A) including a cosmetic agent (a),
a container (B) including a cosmetic agent (b), and
a container (C) containing a cosmetic agent (c),
which are produced separately from each other, wherein
agent (a) in container (A) comprises:
one or more sulfur-containing reductants chosen from the group of (H₂N)(NH)C(SO₂H) formamidine sulfinic acid, sodium dithionite, zinc dithionite, N(CH₂SO₂Na)₃ trisodium [bis(sulfinatomethyl)amino]methanesulfinate, N(CH₂SO₂K)₃ tripotassium [bis(sulfinatomethyl)amino]methanesulfinate, or N(CH₂SO₂H)₃ [bis(sulfinomethyl)amino]methanesulfinic acid, wherein the one or more sulfur-containing reductants are present in a total amount of from about 35.0 to about 55.0 wt %, with respect to the total weight of agent (a),
one or more fatty constituents present in a total amount of from about 5.0 to about 15.0 wt %, with respect to the total weight of agent (a),
wherein agent (a) in container (A) has a water content of at most about 1.0 wt %, with respect to the total weight of agent (a),
agent (b) in container (B) comprises:
one or more metal salts chosen from the group of tin(II) chloride, tin(II) bromide, tin(II) fluoride, tin(II) acetate, tin(II) gluconate, tin(II) lactate, or tin(II) tartrate, wherein the one or more metal salts are present in a total amount of from about 3.0 to about 10.0 wt %, with respect to the total weight of agent (b),
wherein agent (b) in container (B) has a water content of from about 70 to about 93.0 wt %, with respect to the total weight of agent (b), and
agent (c) in container (C) comprises:
one or more acids or one or more alkalizing agents,
wherein agent (c) has a water content of from about 70 to about 99.0 wt %, with respect to the total weight of agent (c).
